(19) United States
(12) Patent Application Publication
(10) Pub. No.: US 2021/0369860 A1
(43) Pub. Date: Dec. 2, 2021

(54) FUNCTIONALIZED NANOPARTICLE FORMULATIONS FOR ORAL DRUG DELIVERY

(71) Applicant: THE REGENTS OF THE UNIVERSITY OF COLORAD, Denver, CO (US)

(72) Inventors: Thomas ANCHORDOQUY, Lakewood, CO (US); Michael GRANER, Aurora, CO (US); Jamie BETKER, Denver, CO (US); Brittany ANGLE, Golden, CO (US)

(21) Appl. No.: 17/288,104

(22) PCT Filed: Oct. 24, 2019
(86) PCT No.: PCT/US2019/057898

§ 371 Date: Apr. 23, 2021

Related U.S. Application Data
(60) Provisional application No. 62/749,765, filed on Oct. 24, 2018.

(51) Int. Cl. A61K 47/69; A61K 47/68; A61K 31/4745; A61K 47/64

(52) U.S. Cl. CPC A61K 47/6931; A61K 47/6849; A61K 31/704; A61K 47/643; A61K 31/4745

(57) ABSTRACT

Drug delivery systems comprising binding partners conjugated to a nanoparticle encapsulating a therapeutic agent formulated for oral administration, and methods of delivering therapeutic agents across the gastrointestinal epithelium.

### STATEMENT OF FEDERALLY SPONSORED RESEARCH

This invention was made with government support under grant numbers GM129046 and EB016378 awarded by the National Institutes of Health. The U.S. government has certain rights in the invention.

### TECHNICAL FIELD

This invention relates generally to the field of drug delivery systems. The system includes the use of FcRn binding partners as targeting moieties conjugated to a nanoparticle for oral administration of therapeutic agents.

### BACKGROUND

Many drugs are not readily absorbed from the gastrointestinal tract, and therefore are typically administered by intravenous infusion. Accordingly, many patients undergoing therapy for cancer and microbial infection receive multiple rounds of infusion as part of their treatment regimen. Because these drugs require infusion, sterile formulations must be prepared and administered by trained personnel. This significantly increases the cost associated with therapy, and the expenses associated with infusion of sterile solutions represents tens of billions of dollars each year. In addition to the higher costs, infusion therapy forces some patients to leave the comfort of their home and be exposed to other sick patients during treatment. As many of these patients are elderly and immune-suppressed, the risk of contracting hospital-acquired infections increases dramatically, which greatly compromises patient outcomes, significantly enhances the chances of mortality, and further increases costs.

There is currently wide interest in the development of nanoparticles for drug delivery, particularly for the protection of acid labile drugs that cannot be administered orally as the free drug. This is particularly relevant to protein and nucleic acid anti-cancer or anti-microbial drugs. For a drug to be orally bioavailable, the molecule must be inherently chemically-resistant or protected from the low pH and degradative enzymes encountered in the stomach. Although encapsulation in a particulate delivery system can protect therapeutics from this harsh environment, absorption of the drug-containing particle from the gut and delivery into the circulation remains a formidable barrier. Thus, there is a desire for an oral delivery system that can protect acid labile drugs from degradation following oral administration but simultaneously overcome the low oral absorption associated with these nanoparticle delivery systems.

### SUMMARY

Compositions and methods are provided for the oral administration of therapeutic agents. In the methods of this disclosure, a therapeutic agent, including for example low-molecular weight compounds, such as small molecules, proteins or nucleic acid therapeutics, such as antibodies, antibody drug conjugates, or gene therapy constructs, cells or microbes, such as stem cells, or recombinant viruses, are encapsulated in nanoparticles comprising a polymer or lipid membrane, e.g. a liposomal structure. These nanoparticles comprise one or more FcRn binding partners conjugated to the lipid or polymer surface to form functionalized particles that can be transported across a cell or cellular layer by receptor mediated transport.

The FcRn binding partner(s) may be an IgG Fc fragment or functional variants thereof that allow the nanoparticle to bind to receptors associated with epithelial cell uptake by transcytosis, such as the FcRn receptor or binding complex that includes the FcRn receptor. This binding can significantly enhance uptake of the nanoparticles from the gastrointestinal tract. The FcRn binding partners may be covalently linked to the surface of the nanoparticles. Alternatively or additionally, the FcRn binding partners may be non-covalently associated with the surface of the nanoparticles.

The nanoparticle of this disclosure may be formed as an extracellular vesicle (e.g. exosomes, ectosome), an endosome, a liposome, a lipoplex, a micelle, or a reverse micelle. Nanoparticles of this disclosure may be used to form compositions comprising the nanoparticles suspended in a pharmaceutically acceptable excipient, and can be administered orally to a subject.

The nanoparticles of this disclosure are also useful for oral or local delivery of a diagnostic agent (e.g., fluorescent or radiopaque compound) or therapeutic agent (e.g., a drug or chemical), delivery vehicle, protein, polynucleotide, and/or combinations thereof across an epithelial barrier into the systemic circulation. These nanoparticles may therefore be used to administer a therapeutic agent to elicit a beneficial effect. The nanoparticles conjugated to the FcRn binding partners are designed to deliver a wide variety of therapeutics, including stem cells, RNA and DNA nucleotides, peptides, carbohydrates, and/or small molecules or chemical compounds. The encapsulated therapeutic agent may be a chemotherapeutic agent, or an antimicrobial agent. In an exemplary embodiment, the therapeutic agent is the topoisomerase inhibitor SN38.

This disclosure therefore provides an FcRn binding partner (e.g., Fc fragment) conjugated to a nanoparticle drug delivery system (e.g., polymeric particles such as nanoparticles or microparticles; liposomes; lipoplexes; genetically engineered viral particles; inorganic particles; etc.) that can transfer the nanoparticles with their therapeutic agents across epithelial cell layers via a transcytosis mechanism. This disclosure includes pharmaceutical compositions that include these nanoparticles, methods of preparing the nanoparticles and compositions containing them, and methods for their use.

This disclosure also provides methods of conjugating the FcRn binding partner (e.g., Fc fragment) to a nanoparticle drug delivery system. For example, the invention provides methods of conjugating an FcRn binding partner (e.g., Fc fragment) to a lipid or polymeric drug delivery nanoparticle. Any isotypes of IgG and IgG Fc fragments may be used. The Fc fragment may be modified. For example, an Fc fragment that is at least 50%, at least 60%, at least 70%, at least 80%, at least 90%, at least 95%, at least 98%, or at least 99% homologous to a human IgG Fc fragment and binds the FcRn receptor may be conjugated to the nanoparticles. The Fc fragment can be attached to a particle using any means known in the art. For example, the attachment may be a covalent attachment (e.g., an amide, an ester, disulfide, or other “click” chemistry), which may optionally comprise a linker (e.g., a peptide linker). The attachment may be an activated ester on the particle and allowed to react with a nucleophile such as a primary amine (e.g., terminal amine, lysine) of the Fc fragment. The attachment may also be non-covalent based on affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

This disclosure also provides kits for the use of the inventive drug delivery systems. Kits may include one or more doses of a drug delivery system for administration to a subject. In certain embodiments, a kit includes a device for delivering the drug delivery system including a syringe, a needle, a catheter, tubing, solutions, buffers, etc. A kit typically includes instructions for administering drug delivery systems. The convenient packaging of a kit allows for the easy use of the drug delivery system or pharmaceutical compositions thereof.

### BRIEF DESCRIPTION OF FIGURES

FIG. 1A is a transmission electron micrograph of exosomes isolated from milk. FIG. 1B shows that bovine milk exosomes contain standard markers of exosome-like vesicles. ExoCheck Array was probed with lysed milk exosomes, showing positive staining for typical exosome markers (CD63, CD81, ANXA5, FLOT1, ICAM, and TSG101) while negative for the putative contaminant GM130. “+cont”=positive control for secondary antibody reactivity; “−cont”=negative control spot.

FIGS. 2A-2F show the absorption from the gastrointestinal tract after oral gavage. Blood samples collected from mice at 30, 60, 120, 240, and 360 min were applied to a membrane (FIG. 2A) and imaged with an infrared detector (FIGS. 2B-2F). Samples from mice administered PBS (FIG. 2B) had no detectable levels of IR signal. Blood from mice administered DIR-labelled liposomes (FIG. 2C) showed low levels of IR signal which was undetectable after 120 min. In contrast, blood from mice administered DIR-labelled milk exosomes (FIGS. 2D-2F) exhibited high IR signal which saturated the detector even on the lowest sensitivity. High signal is maintained in blood for the duration of the experiment (6 h).

FIGS. 3A-3E show the tissue accumulation after oral administration. Organs harvested (6 h) from mice administered PBS (FIG. 3A), DIR-labelled liposomes (FIG. 3B), and DIR-labelled milk exosomes (FIGS. 3C-3E) were imaged. High accumulation of dye in all organs from mice administered exosomes was observed.

FIG. 4 shows the bioavailability in tumor-bearing mice. Blood from mice administered free DIR or DIR-labelled milk exosomes via IV injection is compared to that from mice administered DIR-labelled exosomes (untargeted or targeted w/iRGD) via oral gavage after 4 h. Note that the IV dose was 10% of the oral dose.

FIG. 5 shows the effects of targeting and route of administration on tumor accumulation. Tumors from mice administered free DIR or labelled exosomes via IV injection accumulated comparable levels of delivery to oral administration. Note that IV doses were 10% of that administered orally. iRGD-targeting significantly increased tumor delivery. (n=3 for oral administration; std. error <2%).

FIG. 6 shows double-labelled exosomes isolated from blood after oral administration. The image shows that the membrane dye (left) and the RNA dye (middle) are present in the same exosomes (merged, right) after oral administration.

FIG. 7 shows the effect of incorporating the iRGD ligand on organ accumulation. Relative infrared signal in different organs after intravenous injection of untargeted and targeted exosomes (left panel). IV-administered free DIR and untargeted exosomes compared to orally-administered targeted exosomes (right panel). Note difference in scales between panels.

FIG. 8 shows that co-administration of IgG reduces absorption. DIR-labelled exosomes were co-administered with different amounts of bovine IgG via oral gavage and blood samples were imaged at 30, 60, 120, 180, and 240 minutes (left panel). Co-administration of erythropoietin (EPO) did not reduce absorption (right panel).

FIG. 9 shows a Western blot demonstrating that bovine IgG is present in/on cow milk exosomes. Purified milk exosomes were untreated (XOQ ppt) or treated to strip antibodies (XOs strip). Eluted antibodies (eluted Abs), bovine g-globulin as a positive control, and “Unbound” Abs from the supernatant are also shown.

FIG. 10 shows a chromatogram of the eluate from a sepharose column loaded with exosomes complexed with SN38, demonstrating quantification of SN38 by UPLC.

FIGS. 11A-11D show the concentration of exosome particles per retentate for each of the preparations 8-1, 8-2, 8-3, and 8-4, respectively, of isolated exosomes by tangential flow filtration.

FIGS. 12A-12D show the concentration of exosome particles verses concentration of protein for each of the preparations 8-1, 8-2, 8-3, and 8-4, respectively, of isolated exosomes by tangential flow filtration.

FIGS. 13A-13D show the concentration of protein in the permeate for each of the preparations 8-1, 8-2, 8-3, and 8-4, respectively, of isolated exosomes by tangential flow filtration.

FIGS. 14A-14D show the concentration of protein in the retentate for each of the preparations 8-1, 8-2, 8-3, and 8-4, respectively, of isolated exosomes by tangential flow filtration.

FIGS. 15A-15D show the average particle size of isolated exosomes for each of the preparations 8-1, 8-2, 8-3, and 8-4, respectively, of isolated exosomes by tangential flow filtration.

FIG. 16 shows the mass of SN38 (pg) per elution number comparing preparations made with and without 10 mM phosphate buffer, pH 5.

FIG. 17 shows the mass of SN38 (pg) per elution number.

FIG. 18 shows the mass of SN38 (pg) per elution number by exosomes prepared by tangential flow filtration from preparation 8-2 in Example 7.

FIG. 19 shows the number of exosome particles obtained, normalized to starting milk volume, comparing recovery by ultracentrifugation verses recovery by tangential flow filtration.

FIG. 20 shows protein concentration (mg/ml) verses particle concentration (particles/ml), comparing results of ultracentrifugation verses tangential flow filtration for exosome preparation.

FIG. 21 shows the mass of doxorubicin (pg) recovered per elution number as described in Example 8.

### DETAILED DESCRIPTION

This invention is not limited to the methodology, protocols, cell lines, animal species or genera, and reagents described, as such may vary. The terminology used herein is for describing particular embodiments only and is not intended to limit the scope of the present invention, which will be limited only by the appended claims.

As used herein the singular forms “a”, “an”, and “the” include plural referents unless the context clearly dictates otherwise. Thus, for example, reference to “a cell” includes a plurality of such cells and equivalents thereof known to those skilled in the art.

All technical and scientific terms used herein have the same meaning as commonly understood to one of ordinary skill in the art to which this invention belongs unless clearly indicated otherwise.

The term “liposome” refers to a microscopic closed vesicle having an internal phase enclosed by lipid bilayer. A liposome can be a small single-membrane liposome such as a small unilamellar vesicle (SUV), large single-membrane liposome such as a large unilamellar vesicle (LUV), a still larger single-membrane liposome such as a giant unilamellar vesicle (GUV), a multilayer liposome having multiple concentric membranes such as a multilamellar vesicle (MLV), or a liposome having multiple membranes that are irregular and not concentric such as a multivesicular vesicle (MVV). Additional description of well-known liposome forms is provided in: U.S. Pat. Publication No. 2012/0128757; U.S. Pat. Nos. 4,235,871; 4,737,323; WO 96/14057; New, et al., (1990) Liposomes: A practical approach, IRL Press, Oxford, pages 33-104; and Lasic et al., (1993) Liposomes from physics to applications, Elsevier Science Publishers BV, Amsterdam.

The term “encapsulate” and grammatical variations thereof, refers to therapeutic agent that is associated with the surface of, encapsulated within, complexed with, forming a complex with, surrounded by, embedded in an external layer, and/or dispersed throughout the nanoparticles of this disclosure.

The term “nanoparticle internal phase” refers to a region enclosed within the external layer of the nanoparticle (e.g., within the lipid bilayer of a liposome). By contrast, the term “nanoparticle external phase” refers to the region not enclosed by the external layer of the nanoparticle (e.g., not within the lipid bilayer of a liposome), such as the region apart from the internal phase and the lipid bilayer in the case where the liposome is dispersed in liquid.

The term “target” or “marker,” as used herein, refers to any entity that is capable of specifically binding to a targeting moiety (e.g., FcRn binding partner, Fc fragment, etc.). In some embodiments, targets are specifically associated with one or more tissue types. In some embodiments, targets are specifically associated with one or more cell types. Detection or measurement of a cell type specific marker may make it possible to distinguish the cell type or types of interest from cells of many, most, or all other types. A target can comprise a protein, a carbohydrate, a lipid, and/or a nucleic acid.

A substance is considered to be “targeted” for the purposes of this disclosure it specifically binds to a targeting moiety (e.g., FcRn binding partner, Fc fragment, etc.). In some embodiments, a targeting moiety (e.g., FcRn binding partner, Fc fragment, etc.) specifically binds to its target under stringent conditions. An inventive drug delivery conjugate comprising a targeting moiety (e.g., FcRn binding partner, Fc fragment, etc.) is considered to be “targeted” if the targeting moiety specifically binds to a target, thereby delivering the entire drug delivery conjugate composition to a specific organ, tissue, cell, and/or subcellular locale.

The term “targeting moiety,” as used herein, refers to any moiety that binds to a component associated with a cell. Such a component is referred to as a “target” or a “marker.” A targeting moiety may be a polypeptide, glycoprotein, nucleic acid, small molecule, carbohydrate, lipid, etc. A targeting moiety may be an antibody or functional portion thereof.

The term “therapeutic agent” or “drug,” as used herein, refers to an agent that is administered to a subject to treat a disease, disorder, or other clinically recognized condition that is harmful to the subject, or for prophylactic purposes, and has a clinically significant effect on the body to treat or prevent the disease, disorder, or condition. Therapeutic agents include, without limitation, agents listed in the United States Pharmacopeia (U.S.P.), Goodman and Gilman's The Pharmacological Basis of Therapeutics, 10.sup.th Ed., McGraw Hill, 2001; Katzung, Ed., Basic and Clinical Pharmacology, McGraw-Hill/Appleton & Lange, 8.sup.th ed., Sep. 21, 2000; Physician's Desk Reference (Thomson Publishing); and/or The Merck Manual of Diagnosis and Therapy, 18.sup.th ed., 2006, Beers and Berkow, Eds., Merck Publishing Group; or, in the case of animals, The Merck Veterinary Manual, 9.sup.th ed., Kahn, Ed., Merck Publishing Group, 2005.

As used in this disclosure, “conjugated” means two entities (e.g., a tumor targeting peptide and a reporting agent) are associated with sufficient affinity that the therapeutic/diagnostic benefit of the association between the two entities is realized. Conjugation can be achieved by covalent or non-covalent bonding, as well as by other forms of association, such as entrapment of one entity on or within the other.

### Nanoparticles

There are no limitations on the nanoparticles of this disclosure so long as they can encapsulate therapeutic agents and associate, covalently or non-covalently, with FcRn binding partner(s) that bind with a FcRn receptor or receptor complex including a FcRn receptor, and allow the nanoparticles to cross a cell layer by transcytosis. Preferably, the nanoparticles are suitable for oral administration. Additionally, the nanoparticles preferably exhibit in vitro and in vivo stability.

The membrane constituents of liposomal nanoparticles may include phospholipids and/or phospholipid derivatives. Representative examples of such phospholipids and phospholipid derivatives include, without limitation, phosphatidyl ethanolamine, phosphatidyl choline, phosphatidyl serine, phosphatidyl inositol, phosphatidyl glycerol, cardiolipin, sphingomyelin, ceramide phosphoryl ethanolamine, ceramide phosphoryl glycerol, ceramide phosphoryl glycerol phosphate, 1,2-dimyristoyl-1,2-deoxyphosphatidyl choline, plasmalogen, and phosphatidic acid. It is also acceptable to combine one or more of these phospholipids and phospholipid derivatives.

The fatty-acid residues in these phospholipids and phospholipid derivatives and may include saturated or unsaturated fatty-acid residues having a carbon chain length of 8, 9, 10, 11, 12, 13, 14, 15, 16, 17, 18, 19, 20, or longer. Representative, non-limiting examples include acyl groups derived from fatty-acids such as lauric acid, myristic acid, palmitic acid, stearic acid, oleic acid, and linoleic acid. Phospholipids derived from natural substances such as egg-yolk lecithin and soy lecithin, partially hydrogenated egg-yolk lecithin, (completely) hydrogenated egg-yolk lecithin, partially hydrogenated soy lecithin, and (completely) hydrogenated soy lecithin whose unsaturated fatty-acid residues are partially or completely hydrogenated, and the like, may also be used.

The mixing amount (mole fraction) of the phospholipids and/or phospholipid derivatives that are used when preparing the liposome may be between 10% to 80% relative to the entire liposome membrane composition can be used.

In addition to phospholipids and/or phospholipid derivatives, the liposomal nanoparticles can further include sterols, such as cholesterol and cholestanol as membrane stabilizers and fatty acids having saturated or unsaturated acyl groups, such as those having a carbon number of 8, 9, 10, 11, 12, 13, 14, 15, 16, 17, 18, 19, 20, or longer. There are no limitations on the amount (mole fraction) of these sterols that are used when preparing a liposome, but 1 to 60% relative to the entire liposome membrane composition is preferable. Similarly, there are no limitations on the amount (mole fraction) of the fatty acids, but 0 to 30% relative to the entire liposome membrane composition is preferable. With respect to the amount (mole fraction) of antioxidants, it is sufficient if an amount is added that can obtain the antioxidant effect, but 0 to 15% of the entire liposome membrane composition is preferable.

The liposomal nanoparticles may also contain functional lipids and modified lipids as membrane constituents. Representative, non-limiting examples of functional lipids include lipid derivatives retained in blood (e.g., glycophorin, ganglioside GM1, ganglioside GM3, glucuronic acid derivatives, glutamic acid derivatives, polyglycerin phospholipid derivatives, polyethylene glycol derivatives (methoxypolyethylene glycol condensates, etc.) such as N-[carbonyl-methoxy polyethylene glycol-2000]-1,2-dipalmitoyl-sn-glycero-3-phosphoethanolamine, N-[carbonyl-methoxy polyethylene glycol-5000]-1,2-dipalmitoyl-sn-glycero-3-phosphoethanolamine, N-[carbonyl-methoxy polyethylene glycol-750]-1,2-distearoyl-sn-glycero-3-phosphoethanolamine, N-[carbonyl-methoxy polyethylene glycol-2000]-1,2-distearoyl-sn-glycero-3-phosphoethanolamine (MPEG 2000-distearoyl phosphatidyl ethanolamine), and N-[carbonyl-methoxy polyethylene glycol-5000]-1,2-distearoyl-sn-glycero-3-phosphoethanolamine, which are condensates of phosphoethanolamine and methoxy polyethylene glycol), temperature-sensitive lipid derivatives (e.g., dipalmitoyl phosphatidylcholine), pH-sensitive lipid derivatives (e.g., dioleoyl phosphatidyl ethanolamine), and the like. Liposomes containing lipid derivatives retained in blood are useful for improving the blood retention of the liposome, because the liposome becomes difficult to capture in the liver as a foreign impurity. Similarly, liposomes containing temperature-sensitive lipid derivatives are useful for causing destruction of liposome at specific temperatures and/or causing changes in the surface properties of the liposome. Furthermore, by combining this with an increase in temperature at the target site, it is possible to destroy the liposome at the target site, and release the therapeutic agent at the target site. Liposomes containing pH-sensitive lipid derivatives are useful for enhancing membrane fusion of liposome and endosome when the liposome is incorporated into cells due to the endocytosis to thereby improve transmission of the therapeutic agent to the cytoplasm.

Representative examples of modified lipids include PEG lipids, sugar lipids, antibody-modified lipids, peptide-modified lipids, and the like. Liposomes containing such modified lipids can be targeted to desired target cells or target tissue.

Based on the description above and well-known methods in the art, the composition of the liposome membrane constituents having such membrane permeability at a level allowing practical application can be appropriately selected by those skilled in the art according to the therapeutic agent, compatibility with, or functionalization by, the FcRn binding partners, and optionally, other targeting agents.

The nanoparticles of this disclosure may also be polymeric particles. A wide variety of polymers and methods for forming nanoparticles therefrom are known in the art of drug delivery. Thus, the matrix of a nanoparticle of this disclosure may comprise one or more polymers. Any polymer may be used in accordance with the present invention. Polymers may be natural or unnatural (synthetic) polymers. Polymers may be homopolymers or copolymers comprising two or more monomers. In terms of sequence, copolymers may be random, block, or comprise a combination of random and block sequences. Typically, polymers in accordance with this disclosure are organic polymers.

Examples of suitable polymers include polyalkylenes (e.g., polyethylenes), polycarbonates (e.g., poly(1,3-dioxan-2one)), polyanhydrides (e.g., poly(sebacic anhydride)), polyhydroxyacids (e.g., poly(β-hydroxyalkanoate)), polyfumarates, polycaprolactones, polyamides (e.g., polycaprolactam), polyacetals, polyethers, polyesters (e.g., polylactide, polyglycolide), poly(orthoesters), polyvinyl alcohols, polyurethanes, polyphosphazenes, polyacrylates, polymethacrylates, polycyanoacrylates, polyureas, polystyrenes, polyamines, poly(arylates), polycarbonates, poly(propylene fumarates), polyhydroxyalkanoates, polyketals, polyesteramides, poly(dioxanones), polyhydroxybutyrates, polyhydroxyvalyrates, polyorthocarbonates, poly(vinyl pyrrolidone), polyalkylene oxalates, polyalkylene succinates, poly(malic acid), poly(methyl vinyl ether), and poly(maleic anhydride). In some embodiments, polymers in accordance with the present invention include polymers which have been approved for use in humans by the United States Food and Drug Administration (U.S.F.D.A.) under 21 C.F.R. § 177.2600, including but not limited to polyesters (e.g., polylactic acid, polyglycolic acid, poly(lactic-co-glycolic acid)), polycaprolactone, polyvalerolactone, poly(1,3-dioxan-2one)); polyanhydrides (e.g., poly(sebacic anhydride)); polyethers (e.g., polyethylene glycol); polyurethanes; polymethacrylates; polyacrylates; and polycyanoacrylates.

The polymers can be hydrophilic. For example, polymers may comprise anionic groups (e.g., phosphate group, sulphate group, carboxylate group); cationic groups (e.g., quaternary amine group); or polar groups (e.g., hydroxyl group, thiol group, amine group).

Polymers may be modified with one or more moieties and/or functional groups in addition to the FcRn binding partner. Any moiety or functional group can be used in accordance with the present invention. Polymers may be modified with polyethylene glycol (PEG), with a carbohydrate, and/or with acyclic polyacetals derived from polysaccharides (Papisov, 2001, ACS Symposium Series, 786:301).

Polymers may be modified with a lipid or fatty acid group. A fatty acid group may be one or more of butyric, caproic, caprylic, capric, lauric, myristic, palmitic, stearic, arachidic, behenic, or lignoceric acid. In some embodiments, a fatty acid group may be one or more of palmitoleic, oleic, vaccenic, linoleic, alpha-linoleic, gamma-linoleic, arachidonic, gadoleic, arachidonic, eicosapentaenoic, docosahexaenoic, or erucic acid.

Polymers may be polyesters, including copolymers comprising lactic acid and glycolic acid units, such as poly(lactic acid-co-glycolic acid) and poly(lactide-co-glycolide), collectively referred to herein as “PLGA”; and homopolymers comprising glycolic acid units, referred to herein as “PGA,” and lactic acid units, such as poly-L-lactic acid, poly-D-lactic acid, poly-D,L-lactic acid, poly-L-lactide, poly-D-lactide, and poly-D,L-lactide, collectively referred to herein as “PLA.” In some embodiments, exemplary polyesters include, for example, polyhydroxyacids; lactide-PEG copolymers (e.g., PLA-PEG copolymers); glycolide-PEG copolymers (e.g., PGA-PEG copolymers); copolymers of lactide and glycolide (e.g., PLGA); copolymers of lactide, glycolide, and PEG (e.g., PLGA-PEG copolymers); and derivatives thereof In some embodiments, polyesters include, for example, polyanhydrides, poly(ortho ester), poly(ortho ester)-PEG copolymers, poly(caprolactone), poly(caprolactone)-PEG copolymers, polylysine, polylysine-PEG copolymers, poly(ethylene imine), poly(ethylene imine)-PEG copolymers, poly(L-lactide-co-L-lysine), poly(serine ester), poly(4-hydroxy-L-proline ester), poly[α-(4-aminobutyl)-L-glycolic acid], and derivatives thereof.

The polymer may comprise PLGA. PLGA is a biocompatible and biodegradable co-polymer of lactic acid and glycolic acid, and various forms of PLGA are characterized by the ratio of lactic acid:glycolic acid. Lactic acid can be L-lactic acid, D-lactic acid, or D,L-lactic acid. The degradation rate of PLGA can be adjusted by altering the lactic acid:glycolic acid ratio. In some embodiments, PLGA to be used in accordance with the present invention is characterized by a lactic acid:glycolic acid ratio of approximately 85:15, approximately 75:25, approximately 60:40, approximately 65:35, approximately 50:50, approximately 40:60, approximately 25:75, or approximately 15:85.

Polymers may be one or more acrylic polymers. Acrylic polymers include, for example, acrylic acid and methacrylic acid copolymers, methyl methacrylate copolymers, ethoxyethyl methacrylates, cyanoethyl methacrylate, aminoalkyl methacrylate copolymer, poly(acrylic acid), poly(methacrylic acid), methacrylic acid alkylamide copolymer, poly(methyl methacrylate), poly(methacrylic acid anhydride), methyl methacrylate, polymethacrylate, poly(methyl methacrylate) copolymer, polyacrylamide, aminoalkyl methacrylate copolymer, glycidyl methacrylate copolymers, polycyanoacrylates, and combinations comprising one or more of the foregoing polymers. The acrylic polymer may comprise fully-polymerized copolymers of acrylic and methacrylic acid esters with a low content of quaternary ammonium groups.

Polymers can be cationic polymers. In general, cationic polymers can condense and/or protect negatively-charged strands of nucleic acids (e.g., DNA, RNA, or derivatives thereof). Amine-containing polymers such as poly(lysine) (Zauner et al., 1998, Adv. Drug Del. Rev., 30:97; and Kabanov et al., 1995, Bioconjugate Chem., 6:7), poly(ethylene imine) (PEI; Boussif et al., 1995, Proc. Natl. Acad. Sci., USA, 1995, 92:7297), and poly(amidoamine) dendrimers (Kukowska-Latallo et al., 1996, Proc. Natl. Acad. Sci., USA, 93:4897; Tang et al., 1996, Bioconjugate Chem., 7:703; and Haensler et al., 1993, Bioconjugate Chem., 4:372) are positively-charged at physiological pH, form ion pairs with nucleic acids, and mediate transfection in a variety of cell lines.

Polymers can be degradable polyesters bearing cationic side chains that are less toxic than poly(lysine) and PEI, and degrade into non-toxic metabolites.

Polymers can be anionic polymers. Anionic polymers comprise carboxyl, sulfate, or groups. Anionic polymers include, but are not limited to, dextran sulfate, heparan sulfate, alginic acid, polyvinylcarboxylic acid, arabic acid carboxymethylcellulose, and the like. Anionic polymers may be provided as a salt (e.g., sodium salt).

The properties of these and other polymers and methods for preparing them are well known in the art (see, for example, U.S. Pat. Nos. 6,123,727; 5,804,178; 5,770,417; 5,736,372; 5,716,404; 6,095,148; 5,837,752; 5,902,599; 5,696,175; 5,514,378; 5,512,600; 5,399,665; 5,019,379; 5,010,167; 4,806,621; 4,638,045; and U.S. Pat. No. 4,946,929; Wang et al., 2001, J. Am. Chem. Soc., 123:9480; Lim et al., 2001, J. Am. Chem. Soc., 123:2460; Langer, 2000, Acc. Chem. Res., 33:94; Langer, 1999, J. Control. Release, 62:7; and Uhrich et al., 1999, Chem. Rev., 99:3181). More generally, a variety of methods for synthesizing suitable polymers are described in Concise Encyclopedia of Polymer Science and Polymeric Amines and Ammonium Salts, Ed. by Goethals, Pergamon Press, 1980; Principles of Polymerization by Odian, John Wiley & Sons, Fourth Edition, 2004; Contemporary Polymer Chemistry by Allcock et al., Prentice-Hall, 1981; Deming et al., 1997, Nature, 390:386; and in U.S. Pat. Nos. 6,506,577, 6,632,922, 6,686,446, and 6,818,732.

The polymers can be linear or branched polymers. The polymers can be dendrimers. The polymers can be substantially cross-linked to one another. Polymers can be substantially free of cross-links and may be used without undergoing a cross-linking step.

The polymers may be a homopolymer, block copolymer, diblock triblock, multiblock copolymer, linear polymer, dendritic polymer, branched polymer, graft copolymer, blend, mixture, and/or adduct of any of the foregoing and other polymers.

The therapeutic agent(s) are preferably released from the nanoparticles of this disclosure after the particle reaches a target tissue, cell, or intracellular organelle. The nanoparticle compositions of this disclosure contain membrane constituents that are biodegradable, and ultimately decompose in target tissue or the like and that the encapsulated therapeutic agent is thereby released through dilution, chemical equilibrium, and/or enzymatic degradation.

Depending on the desired application, the particle size of the nanoparticle can be regulated. For example, nanoparticles of this disclosure may be in the size range of 30-400 nm in diameter. The term “nanoparticle particle size” refers to the weight-average particle size according to a dynamic light scattering method (e.g., quasi-elastic light scattering method). Nanoparticle sizes can be measured using dynamic light scattering instruments. The instruments measure Brownian motion of the particles and particle size is determined based on established dynamic light scattering.

There are no limitations on the solvent of the nanoparticle internal phase. Exemplary buffer solutions include, without limitation, a phosphate buffer solution, citrate buffer solution, and phosphate-buffered physiological saline solution, physiological saline water, culture mediums for cell culturing, and the like. An exemplary solvent that may suspend the therapeutic agent(s) and form at least a portion of the nanoparticle internal phase is n-methylpyrrolidone. In the case where buffer solution is used as solvent, it is preferable that the concentration of buffer agent be 5 to 300 mM, 10 to 100 mM, or any range in between. There are also no limitations on the pH of the liposome internal phase. The liposome internal phase may have a pH between 2 and 11.

### FcRn Binding Partners

The neonatal Fc receptor (FcRn) is a well characterized Fc receptor similar in structure to the MHC class I molecule that was first discovered in rodents as a unique receptor capable of transporting IgG from mother's milk across the epithelium of newborn rodent's gut into the newborn's bloodstream. It has also been shown to play a role in IgG and serum albumin turnover. The FcRn receptor binds IgG (but not other immunoglobulin classes such as IgA, IgD, IgM, and/or IgE) at acidic pH and not at basic pH. FcRn transports IgG across epithelial cells either in the direction of apical to basolateral surface or in the direction of basolateral to apical surface. FcRn binding partners may include whole IgG, the Fc fragment of IgG, and/or other fragments of IgG that include the complete binding region for the FcRn receptor. The region of the Fc portion of IgG that binds to the FcRn receptor has been described based upon X-ray crystallography (Burmeister, et al., 1994, Nature, 372:379). The major contact area of Fc with the FcRn receptor is near the junction of the CH2 and CH3 domains. The Fc-FcRn contacts are all within a single Ig heavy chain. The Fc region of IgG can be modified to form Fc fragments, which will be bound by the FcRn receptor and/or other receptors that participate in the receptor mediated endocytosis of the nanoparticles. Such modifications include modifications remote from the FcRn contact sites as well as modifications within the contact sites that preserve or even enhance binding. By using FcRn targeted nanoparticles, it may be possible to enhance delivery across cells, layers of cells, and/or tissues, resulting in improved drug distribution and targeting.

An FcRn binding partner means any entity (e.g., peptides, glycopeptides, proteins, glycoproteins, polynucleotides, aptamers, spiegelmers, antibodies (e.g., monoclonal antibodies), antibody fragments, small molecule ligands, carbohydrate ligands, nanobodies, avimers, metal complexes, etc.) that can be specifically bound by the FcRn receptor and/or associated proteins with subsequent active transport of the conjugated nanoparticle and its payload (e.g., particle or agent). As mentioned above, the FcRn receptor has been isolated from several mammalian species, including humans. The sequence of the human FcRn, rat FcRn, and mouse FcRn may be found in Story et al. (1994, J. Exp. Med., 180:2377; incorporated herein by reference). The FcRn receptor molecule actively transports the IgG transcellularly in a luminal to serosal direction, and then releases the IgG at the relatively high pH found in the interstitial fluids. FcRn receptors can be isolated by cloning or by affinity purification using, for example, monoclonal antibodies. Such isolated FcRn receptors then can be used to identify and isolate FcRn binding partners. The FcRn binding partner can be a small molecule, a protein or peptide, an immunoglobulin, a glycoprotein, a polynucleotide (e.g., aptamer, RNAi-inducing entity, etc.), a carbohydrate, a lipid, or any other type of chemical compound. The FcRn binding partner is preferably a protein or peptide. In some embodiments, the FcRn binding partner is an immunoglobulin (e.g. Fc fragment). In some embodiments, it is an aptamer.

An FcRn binding partner may also be an Fc fragment, such as an Fc fragment of an IgG antibody, including any isotype of IgG antibody (e.g., IgG 1, IgG 2, IgG 2a, IgG 2b, IgG 3, IgG 4).

### Targeting Ligands

The nanoparticle drug delivery systems of this disclosure may comprise targeting ligands in addition to the FcRn binding partner. These additional targeting moieties may help direct drug delivery systems to their appropriate targets following absorption from the gastrointestinal tract.

Because expression constructs (i.e., plasmids) that expresses antisense or other nucleic acid therapeutics may be effectively delivered using the lipoplex formulations of this disclosure, it will be important to maximize delivery and retention at the delivery site (e.g., at the tumor for cancer therapies). Although non-targeted nanoparticles of this disclosure can deliver nucleic acid therapeutics at levels resulting in robust tumor expression, the additional incorporation of a targeting ligand in these lipoplexes can increase levels of gene expression in the target tissues. But simply incorporating a ligand into a particle does not necessarily enhance uptake or specificity. In fact, studies have shown that the proteins adsorbed to a nanoparticle after systemic administration can obscure/foul targeting ligands (Hagiwara K, Ochiya T, Kosaka N. A paradigm shift for extracellular vesicles as small RNA carriers: from cellular waste elimination to therapeutic applications. Drug Deliv Transl Re. 2014; 4:31-7.) An approach to avoid ligand fouling that has been extensively studied is the use of PEGylated components. But concerns about PEG immunogenicity and reduced intracellular delivery suggest that superior delivery might be achieved by avoiding the use of PEG in delivery formulations. Indeed, multiple studies have documented that even very low levels of PEGylation (1%) can significantly reduce transfection rates (Lee J, et al Liposome-Based Engineering of Cells to Package Hydrophobic Compounds in Membrane Vesicles for Tumor Penetration. Nano letters. 2015; 15:2938-44; Syn N, et al. Exosome-Mediated Metastasis: From Epithelial-Mesenchymal Transition to Escape from Immunosurveillance. Trends Pharmacol Sci. 2016; 37:606-17; Verhoef J J F and Anchordoquy T J. Questioning the Use of PEGylation for Drug Delivery. Drug Delivery and Translational Res. 3:499-503 (2013) and Xu L and Anchordoquy T J. Effect of cholesterol nanodomains on the targeting of lipid-based gene delivery in cultured cells. Molecular Pharmaceutics 7 (4):1311-17 (2010)). Thus, avoiding PEGylated components is another distinct advantage of the nanoparticle delivery systems of this disclosure.

The use of targeting ligands may require the use of an effective linker to link the chosen ligand(s) to the nanoparticles. The nanoparticles delivery systems of this disclosure may possess a cholesterol domain. This aspect of the nanoparticles imparts a distinct advantage in that undetectable amounts of protein are adsorbed to these domains, making them ideal for presenting targeting ligands. Furthermore, conjugating a targeting ligand to cholesterol preferentially locates the ligand within the protein-free cholesterol domain, which enhances the transfection rates of the nanoparticles of this disclosure, both in vitro and in vivo. Cholesterol membrane domains formed within nanoparticles of this disclosure may endow these nanoparticles with improved serum stability, transfection, and targeting both in vitro and in vivo.

Targeting ligands (in addition to the FcRn binding partner) may help direct drug delivery nanoparticles of this disclosure to their appropriate systemic targets. One of ordinary skill in the art will recognize that any additional targeting moiety which directs the drug delivery system to any target site may be utilized. Exemplary additional targeting moieties include, but are not limited to, proteins (e.g., peptides, antibodies, glycoproteins, polypeptides, etc., or characteristic portions thereof), nucleic acids (e.g. aptamers, Spiegelmers, RNAi-inducing entities, etc., or characteristic portions thereof), carbohydrates (e.g. monosaccharides, disaccharides, polysaccharides, etc., or characteristic portions thereof), lipids or characteristic portions thereof, small molecules or characteristic portions thereof, and viruses.

The targeting molecules described herein can bind to lesions, particularly tumor, cancer tissues/cells and vascular endothelial cells in tumor microenvironment, both in vitro and in vivo. Thus, when targeting molecules are conjugated with a reporting agent (e.g., a fluorescent or radioactive agent in bioimaging), they direct the agent to a cancer site, thereby facilitating cancer diagnosis. Exemplary targeting molecules include small peptides and/or proteins, such as Arg-Gly-Asp (RGD), Asn-Gly-Arg (NGR), cyclic NGR, disulfide-based cyclic RGD (iRGD), Lyp-1, gastrin, bombesin, octreotide, or derivatives thereof. Exemplary proteins include, but not limited to epidermal growth factor (EGF), anti-EGFR antibody, vascular endothelial growth factor (VEGF), anti-VEGFR antibody, anti-HER2 antibody, hepatocyte growth factor receptor (HGFR), anti-HGFR antibody, tumor necrosis factor (TNF), or anti-TNF antibody.

### Therapeutic Agents

There are no limitations on the therapeutic agent encapsulated in the nanoparticles of this disclosure.

As therapeutic agents, any desired agent can be used, such as those useful in the fields of medicines (including diagnostic drugs), cosmetic products, food products, and the like. For example, the therapeutic agent can be selected from a variety of known classes of useful agents, including, for example, proteins, peptides, nucleotides, anti-obesity drugs, nutraceuticals, corticosteroids, elastase inhibitors, analgesics, anti-fungals, oncology therapies, anti-emetics, analgesics, cardiovascular agents, anti-inflammatory agents, anthelmintics, anti-arrhythmic agents, antibiotics (including penicillins), anticoagulants, antidepressants, antidiabetic agents, antiepileptics, antihistamines, antihypertensive agents, antimuscarinic agents, antimycobacterial agents, antineoplastic agents, immunosuppressants, antithyroid agents, antiviral agents, anxiolytic sedatives (hypnotics and neuroleptics), astringents, beta-adrenoceptor blocking agents, blood products and substitutes, cardiac inotropic agents, contrast media, corticosteroids, cough suppressants (expectorants and mucolytics), diagnostic agents, diagnostic imaging agents, diuretics, dopaminergics (antiparkinsonian agents), haemostatics, immunological agents, lipid regulating agents, muscle relaxants, parasympathomimetics, parathyroid calcitonin and biphosphonates, prostaglandins, radio-pharmaceuticals, sex hormones (including steroids), anti-allergic agents, stimulants and anoretics, sympathomimetics, thyroid agents, vasodilators and xanthines. With respect to therapeutic agents, it is acceptable to combine one or more agents.

The therapeutic agents can be low-molecular compounds, such as small molecules. An exemplary therapeutic agent for encapsulation in the nanoparticles of this disclosure is the chemotherapeutic agent 7-Ethyl-10-hydroxycamptothecin (SN38). Alternatively or additionally, the therapeutic agents can be proteins or nucleic acid therapeutics, such as antibodies, antibody drug conjugates, or gene therapy constructs. Alternatively or additionally, the therapeutic agents can be cells or microbes, such as stem cells, or recombinant viruses.

### Nanoparticle Compositions

The term “nanoparticle composition” refers to a composition that contains a nanoparticle of this disclosure functionalized with a FcRn binding partner and encapsulating a therapeutic agent in the internal phase. Nanoparticle compositions can include solid and liquid forms. In the case where the nanoparticle composition is in a solid form, it can be made into a liquid form by dissolving or suspending it in a prescribed solvent. In the case where the nanoparticle composition is frozen solid, it can be made into a liquid form by melting by leaving it standing at room temperature.

The concentration of nanoparticle and the concentration of the therapeutic agent in the nanoparticle composition can be appropriately set according to the nanoparticle composition objective, formulation, and other considerations well known to the skilled artisan. It is preferable that the quantity of the FcRn binding partner(s) in the nanoparticle composition be 0.1 to 1000 mol equivalent relative to the therapeutic agent, and more preferably 1 to 100 mol equivalent relative to the therapeutic agent.

There are no limitations on the solvent of the nanoparticle composition in the case where the composition is a liquid formulation. Representative examples include buffer solutions such as phosphate buffer solution, citrate buffer solution, and phosphate-buffered physiological saline solution, physiological saline water, and culture mediums for cell culturing. Solvents such as n-methylpyrrolidone (NMP) may be used to dissolve or suspend therapeutic agents that are not freely soluble in aqueous solution.

Similarly, there are no limitations on the pH of the nanoparticle external phase of the liposome composition. For example, the pH may be between 2 and 11. Preferably, the pH of the nanoparticle external phase in these compositions is slightly acidic, as FcRn receptor binding is enhanced at acidic pH. Thus, the pH of the external phase of the nanoparticle compositions is preferably a pH between pH 4 and pH 7, or between pH 4.5 and pH 6.5, or at pH of about 5.0, or at pH of about 6.0.

Pharmaceutical excipients may be added to the nanoparticle compositions of this disclosure. Acceptable excipients may include sugar, such as monosaccharides such as glucose, galactose, mannose, fructose, inositol, ribose, and xylose; disaccharides such as lactose, sucrose, cellobiose, trehalose, and maltose; trisaccharides such as raffinose and melezitose; polysaccharides such as cyclodextrin; and sugar alcohols such as erythritol, xylitol, sortibol, mannitol and maltitol; polyvalent alcohols such as glycerin, diglycerin, polyglycerin, propylene glycol, polypropylene glycol, ethylene glycol, diethylene glycol, triethylene glycol, polyethylene glycol, ethylene glycol monoalkylether, diethylene glycol monoalkylether, 1,3-butylene glycol. Combinations of sugar and alcohol can also be used.

For purposes of stable long-term storage of the nanoparticle composition it is preferable to eliminate the electrolyte in the solvent as much as possible. Moreover, from the standpoint of chemical stability of lipids, it is preferable to set the pH of the solvent from acidic to the vicinity of neutral (e.g., pH 3.0 to 8.0), and to remove dissolved oxygen through nitrogen bubbling. Representative examples of liquid stabilizers include, without limitation, normal saline, isotonic dextrose, isotonic sucrose, Ringer's solution, and Hanks' solution. A buffer substance can be added to provide pH optimal for storage stability. For example, pH between about 6.0 and about 7.5, more preferably pH about 6.5, is optimal for the stability of liposome membrane lipids, and provides for excellent retention of the entrapped entities. Histidine, hydroxyethylpiperazine-ethylsulfonate (HEPES), morpholipo-ethylsulfonate (MES), succinate, tartrate, and citrate, typically at 2-20 mM concentration, are exemplary buffer substances. Other suitable carriers include, e.g., water, buffered aqueous solution, 0.4% NaCl, 0.3% glycine, and the like. Protein, carbohydrate, or polymeric stabilizers and tonicity adjusters can be added, e.g., gelatin, albumin, dextran, or polyvinylpyrrolidone. The tonicity of the composition can be adjusted to the physiological level of 0.25-0.35 mol/kg with glucose or a more inert compound such as lactose, sucrose, mannitol, or dextrin. These compositions can be sterilized by conventional sterilization techniques, e.g., by filtration. The resulting aqueous solutions can be packaged for use or filtered under aseptic conditions and lyophilized, the lyophilized preparation being combined with a sterile aqueous medium prior to administration.

Solid formulations of nanoparticle compositions can also include pharmaceutical excipients. For example, such components can include sugars, such as monosaccharides such as glucose, galactose, mannose, fructose, inositol, ribose, and xylose; disaccharides such as lactose, sucrose, cellobiose, trehalose, and maltose; trisaccharides such as raffinose and melezitose; polysaccharides such as cyclodextrin; and sugar alcohols such as erythritol, xylitol, sorbitol, mannitol, and maltitol. More preferable are blends of glucose, lactose, sucrose, trehalose, and sorbitol. Even more preferable are blends of lactose, sucrose, and trehalose that may allow solid formulations to be stably stored over long periods. When frozen, it is preferable that solid formulations contain polyvalent alcohols (aqueous solutions) such as glycerin, diglycerin, polyglycerin, propylene glycol, polypropylene glycol, ethylene glycol, diethylene glycol, triethylene glycol, polyethylene glycol, ethylene glycol monoalkylether, diethylene glycol monoalkylether and 1,3-butylene glycol. With respect to polyvalent alcohols (aqueous solutions), glycerin, propylene glycol, and polyethylene glycol are preferable, and glycerin and propylene glycol are more preferable. By this refers to, it is possible to stably store the solid formulation over long periods. Sugars and polyvalent alcohols can be used in combination.

### Methods of Making Nanoparticles

Methods for making microparticles for delivery of encapsulated agents are described in the literature (see, e.g., Doubrow, Ed., “Microcapsules and Nanoparticles in Medicine and Pharmacy,” CRC Press, Boca Raton, 1992; Mathiowitz et al., 1987, J. Control. Release, 5:13; Mathiowitz et al., 1987, Reactive Polymers, 6:275; and Mathiowitz et al., 1988, J. Appl. Polymer Sci., 35:755). If particles prepared by any of the above methods have a size range outside of the desired range, particles can be sized, for example, using a sieve. Numerous methods are known in the art for preparing liposomes. Representative examples include, without limitation, the lipid film method (Vortex method), reverse phase evaporation method, ultrasonic method, pre-vesicle method, ethanol injection method, French press method, cholic acid removal method, Triton X-100 batch method, Ca²⁺ fusion method, ether injection method, annealing method, freeze-thaw method, and the like.

The various conditions (quantities of membrane constituents, temperature, etc.) in the nanoparticle preparation can be suitably selected according to the preparation method, target composition, particle size, etc.

It is possible to use a substance dissolved in a solvent or a solid substance as the therapeutic agent according to the physical properties of the therapeutic agent. There are no limitations on the solvent. For example, the solvent may be a substance identical to the liposome external phase. An exemplary solvent in which a therapeutic agent is dissolved or suspended is n-methylpyrrolidone (NMP).

The nanoparticle size can be adjusted as necessary. Particle size can be adjusted, for example, by conducting extrusion (extrusion filtration) under high pressure using a membrane filter of regular pore diameter. Particle size adjustment can be conducted at any timing during manufacture of the liposome composition. For example, particle size adjustment can be conducted before introducing the therapeutic agent complexes into the nanoparticle internal phase or after the therapeutic agent complexes have been remotely loaded into the nanoparticle internal phase.

Well-known methods exist for removing any undesired or unincorporated complexes or compositions, such as therapeutic agent not encapsulated within nanoparticles. Representative examples include, without limitation, dialysis, centrifugal separation, tangential flow filtration (TFF) and gel filtration. Dialysis can be conducted, for example, using a dialysis membrane. As a dialysis membrane, one may cite a membrane with molecular weight cut-off such as a cellulose tube or Spectra/Por. With respect to centrifugal separation, centrifugal acceleration any be conducted preferably at 100,000 g or higher, and more preferably at 300,000 g or higher. Gel filtration may be carried out, for example, by conducting fractionation based on molecular weight using a column such as Sephadex or Sepharose.

An active remote loading method may be used to encapsulate a therapeutic agent within a nanoparticle. Generally, the presence of an ionic gradient (e.g., titratable ammonium, such as unsubstituted ammonium ion) in the inner space of a nanoparticle can provide enhanced encapsulation of weak amphiphilic bases, for example, via a mechanism of “active”, “remote”, or “transmembrane gradient-driven” loading. For example, active remote loading can be achieved using a transmembrane pH gradient. The nanoparticle internal and external phases differ in pH by 1-5 pH units. it is also acceptable if the nanoparticle internal and external phases do not substantially have difference in pH (i.e., the nanoparticle external and internal phases have substantially the same pH). The pH gradient can be adjusted using a compound conventionally known in the art used in pH gradient methods. Representative examples include amino acids such as arginine, histidine, and glycine; acids such as ascorbic acid, benzoic acid, citric acid, glutamic acid, phosphoric acid, acetic acid, propionic acid, tartaric acid, carbonic acid, lactic acid, boric acid, maleic acid, fumaric acid, malic acid, adipic acid, hydrochloric acid, and sulfuric acid; salts of the aforementioned acids such as sodium salt, potassium salt, and ammonium salt; and alkaline compounds such as tris-hydroxymethylamino methane, ammonia water, sodium hydride, potassium hydride, and the like.

Many different ions can be used in the ion gradient method. Representative examples include ammonium sulfate, ammonium chloride, ammonium borate, ammonium formate, ammonium acetate, ammonium citrate, ammonium tartrate, ammonium succinate, ammonium phosphate, and the like. Moreover, with respect to the ion gradient method, the ion concentration of the nanoparticle internal phase can be selected appropriately according to the type of the therapeutic agent. A higher ion concentration is preferable and is preferably 10 mM or higher, more preferably 20 mM or higher, even more preferably 50 mM or higher. Either the nanoparticle internal or external phase can have the higher ion concentration according to the type of the therapeutic agent. On the other hand, it is also acceptable if the nanoparticle internal and external phases do not substantially have a difference in ion concentration, i.e., the liposome external and internal phases have substantially the same ion concentration. The ion gradient can also be adjusted by substituting or diluting the liposome external phase.

The membrane permeability of the nanoparticle may be enhanced using well-known methods, including for example, heating or cooling liposome-containing compositions, adding a membrane fluidizer to liposome-containing compositions, and the like. When nanoparticle-containing compositions are heated or cooled, the therapeutic agent can generally be more efficiently introduced into the nanoparticle internal phase by heating to higher temperatures. Specifically, it is preferable to set the temperature of heating taking into consideration the thermal stability of the therapeutic agent and the employed nanoparticle membrane constituents. It is preferable that the temperature be set to the phase transition temperature of a lipid bilayer membrane or higher. The term “phase transition temperature” of the lipid bilayer membrane of a liposome refers to the temperature at which heat absorption starts (the temperature when endothermic reaction begins) in differential thermal analysis of elevated temperatures conditions. In the heating or cooling step, there are no limitations on the time during which the temperature is maintained at or above/below the phase transition temperature, and this may be properly set within a range, for example, of several seconds to 30 minutes. Taking into consideration the thermal stability of the therapeutic agent and lipids as well as efficient mass production, it is desirable to conduct the treatment within a short time, for example a time between 1 to 30 minutes.

It is also possible to enhance nanoparticle membrane permeability by adding a membrane fluidizer to the obtained mixed solution (that is, adding it to the external phase side of the liposome). Representative examples include organic solvents, surfactants, enzymes, etc. that are soluble in aqueous solvents. Representative organic solvents include monovalent alcohols such as ethyl alcohol and benzyl alcohol; polyvalent alcohols such as glycerin and propylene glycol; aprotic polar solvents such as dimethyl sulfoxide (DMSO). Representative surfactants include anionic surfactants such as fatty acid sodium, monoalkyl sulfate, and monoalkyl phosphate; cationic surfactants such as alkyl trimethyl ammonium salt; ampholytic surfactants such as alkyl dimethylamine oxide; and non-ionic surfactants such as polyoxyethylene alkylether, alkyl monoglyceryl ether, and fatty acid sorbitan ester. Those skilled in the art can set the quantity of membrane fluidizer according to the composition of nanoparticle membrane constituents, the membrane fluidizer, and the like, taking into consideration the degree of efficiency of entrapment of the therapeutic agent due to addition of the membrane fluidizer, the stability of the liposome, etc.

In these production methods, the nanoparticle external phase may be adjusted, for example by drying the nanoparticle composition before and/or after encapsulation of the therapeutic agent. The nanoparticle external phase in the composition can be adjusted (replaced, etc.) to make a final nanoparticle composition if it is to be used as a liquid formulation. Where the nanoparticle composition is to be made into a solid preparation, the liquid nanoparticle composition obtained in the above-mentioned introduction step can be dried to make the final solid nanoparticle composition. Freeze drying and spray drying are representative, non-limiting examples of methods for drying the nanoparticle composition. In cases where the nanoparticle composition is a solid preparation, it can be dissolved or suspended in a suitable solvent and used as a liquid formulation. The solvent for use can be appropriately set according to the purpose of use for the nanoparticle composition.

In these methods, excess or “free” FcRn binding partners or complexes or therapeutic agents may all interfere with, or compete for binding with, FcRn receptor complex, thereby preventing or reducing nanoparticle uptake. Therefore, the nanoparticle compositions are preferably adjusted in the external phase to contain less than 10%, or less than 5%, or less than 2%, or less than 1%, or less than 0.1%, or less than 0.01% unconjugated FcRn binding partners, or unencapsulated or unbound therapeutic agent(s).

The nanoparticles may also be isolated or purified from natural sources. One of ordinary skill in the art would understand that to include nanoparticles that naturally occur in milk or other physiological fluids, and this would include all types of extracellular vesicles including exosomes. For example, exosomes may be isolated from milk or colostrum. In these methods, raw milk or colostrum may be subjected to centrifugation, or a series of sequential centrifugations, and/or filtration step(s) (e.g., either centrifugation, ultrafiltration/diafiltration or tangential flow filtration) to isolate the exosomes. In exemplary methods, raw milk or colostrum is centrifuged and/or filtered to remove protein, fat globules, casein debris, microvesicles, and other large particles present in the milk or colostrum from the exosomes. In these methods, the composition of isolated exosomes may be purified to a protein content less than 10%, or less than 5%, or less than 2%, or less than 1%, or less than 0.1%, or less than 0.01%. A particularly problematic protein in these exosome compositions is bovine serum albumin (BSA), which has been shown to bind to FcRn receptor and may therefore significantly reduce binding of the exosomes to FcRn receptor. Thus, in exemplary methods, the composition of isolated exosomes may be purified to a BSA content less than 10%, or less than 5%, or less than 2%, or less than 1%, or less than 0.1%, or less than 0.01%. Similarly, fat globules present in the exosome preparation may interfere with FcRn receptor complex binding, thereby decreasing exosome uptake by endocytosis. Therefore, in exemplary methods, the composition of isolated exosomes may be purified to a fat content outside of the lipid content of the exosome membrane, of less than 10%, or less than 5%, or less than 2%, or less than 1%, or less than 0.1%, or less than 0.01%.

If particles prepared by any of the above methods have a size range outside of the desired range, particles can be sized, for example, using a sieve.

The nanoparticles may be loaded with one or more therapeutic agents by suspending or dissolving the therapeutic agent(s) in a suitable buffer and/or solvent and incubating the nanoparticles in the suspension/solution containing the therapeutic agent(s). Exemplary solvents include n-methylpyrrolidone (NMP) and/or alcohols, such as ethanol. After an incubation time and temperature suitable to load the nanoparticles with the therapeutic agent(s), excess therapeutic agent that is not encapsulated or bound to the nanoparticles is removed from the composition of nanoparticles by any suitable method, such as centrifugation, dialysis, and filtration. In exemplary methods, the nanoparticle composition may contain unbound or unencapsulated (i.e., “free”) drug of less than 10%, or less than 5%, or less than 2%, or less than 1%, or less than 0.1%, or less than 0.01%.

The resulting nanoparticle preparation may be suspended in a pharmaceutical composition for administration to a mammalian subject, or lyophilized, or frozen, or characterized for consistency or purity or drug loading.

### Pharmaceutical Compositions and Methods of Administration

The nanoparticle compositions described herein can be used as a pharmaceutical composition such as a therapeutic composition or a diagnostic composition in the medical field. For example, the nanoparticle composition can be used as a therapeutic composition by incorporating a therapeutic agent and can be used as a diagnostic composition by incorporating contrast agent as the therapeutic agent. The nanoparticle composition can also be used for any number of other purposes, such as a cosmetic product or food additive.

Typically, the nanoparticle pharmaceutical composition of this disclosure is prepared as an oral formulation, either as a liquid solution or suspension. However, solid forms can also be prepared. The composition can also be formulated into an enteric-coated tablet or gel capsule according to known methods in the art.

In the case where the nanoparticle composition of this disclosure is used as a pharmaceutical composition, the nanoparticle composition can be administered orally, or by injection (intravenous, intra-arterial, or local injection), nasally, subcutaneously, by inhalation, or through eye drops, or local injection to a targeted group of cells or organ. These formulations may include tablet, powder, granulation, syrup, capsule, liquid, and the like in the case of oral administration. These formulations may include Injectable, drip infusion, eye drop, ointment, suppository, suspension, lotion, aerosol, plaster, and the like in the case of non-oral administration.

The term “administering” a substance, such as a therapeutic entity to an animal or cell, is intended to refer to dispensing, delivering or applying the substance to the intended target. In terms of the therapeutic agent, the term “administering” is intended to refer to contacting or dispensing, delivering or applying the therapeutic agent to an animal by any suitable route for delivery of the therapeutic agent to the desired location in the animal, including in any way which is medically acceptable which may depend on the condition or injury being treated.

The dosage of the pharmaceutical composition upon administration can differ depending on the type of target disease, the type of the therapeutic agent, as well as the age, sex, and weight of the patient, the severity of the symptoms, along with other factors. The determination of the appropriate dose regimen for any given therapeutic agent encapsulated within the nanoparticle and for a given patient is well within the skill of the medical professional.

### Kits

Kits are also provided for preparing the nanoparticle compositions of this disclosure. The kit can be used to prepare the nanoparticle composition as a therapeutic or diagnostic, which can be used by a physician or technician in a clinical or research setting, or by a patient.

The kit includes a nanoparticle reagent in a solid or a liquid form. If the nanoparticle reagent is in a solid form, the liposome reagent can be dissolved or suspended in an appropriate solvent to obtain the nanoparticle, and a nanoparticle dispersion liquid can be dried to obtain the nanoparticle reagent. If the nanoparticle reagent is in a solid form, the nanoparticle regent can be dissolved or suspended in an appropriate solvent to make the nanoparticle dispersion liquid. When doing so, the solvent is similar to the nanoparticle external phase in the above-mentioned nanoparticle dispersion liquid.

The kit may also contain one or more therapeutic agent(s). The therapeutic agent can be either in a solid or liquid form (a state of dissolved or suspended in a solvent). When using the kit, if the therapeutic agent is in a solid form, it is preferable that it be dissolved or suspended in an appropriate solvent to make a liquid form. The solvent can be appropriately set according to the physical properties and the like of the therapeutic agent and may be made similar to the liposome external phase in the above-mentioned liposome dispersion liquid, for example.

The kit may also contain a nanoparticle composition described herein including directions for use.

The following examples are set forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed to comprise preferred modes for the practice of the invention. It will be appreciated by those of skill in the art that, in light of the present disclosure, numerous modifications and changes can be made in the particular embodiments exemplified without departing from the intended scope of the invention. All such modifications are intended to be included within the scope of the appended claims.

### EXAMPLES

The following methods were used to conduct the experiments described in Examples 1-6, below: Chemicals: Raw milk from different cows was donated by Pam and Jeff Fiorino of Mucca Bella Dairy (Carr, Colo.). DIR (1,1′-Dioctadecyl-3,3,3′,3′-Tetramethylindotricarbo-cyanine Iodide), DiD (1,1′-Dioctadecyl-3,3,3′,3′-Tetramethylindodicarbocyanine) and SYTO13 were obtained from Thermo Fisher Scientific, Carlsbad, Calif. Egg phosphatidylcholine and cholesterol were acquired from Avanti Polar Lipids (Alabaster, Ala.). Bovine IgG was purchased from Pierce Protein Biology (Thermo Fisher, Rockford, Ill.). A cyclic iRGD peptide (CRGDRGPDC) was purchased from Peptides International (Louisville, Ky.), and specifically conjugated to the hydroxyl moiety of cholesterol via a carbamate linker with the terminal amine.

Exosome Isolation and characterization: Exosomes were freshly harvested from raw cow milk by the three-step centrifugation process according to the methods of Agrawal et al. (Nanomed-Nanotechnol 13(5):1627-36 (2017)). Briefly, milk was centrifuged at 13,000×g for 30 min, before passing through cheese cloth. Large fat globules, casein debris, large particles, and microvesicles were then removed by spinning at 100,000×g for 60 min. Exosomes were isolated by centrifugation at 135,000×g for 90 min at 4° C. on a Beckman Optima centrifuge (model #LE-80k, SW-28 rotor, K factor 257.1 at 135,000×g). The size and zeta potential of exosomes was measured on a Zetasizer (Malvern, England). In addition, isolated exosomes were visualized via transmission electron microscopy. Four to five microliters of sample were applied to negatively charged formvar/carbon 300 mesh copper grids (EMS) for 20 seconds, then the excess was wicked off with filter paper. The grid was quickly rinsed by touching it to two drops of MilliQ water, wicking each time, and then stained with two drops of 0.75% uranyl formate. The grid was touched to the first drop of uranyl formate for a few seconds, wicked with filter paper, and then applied to the second drop of uranyl formate for 20 seconds. After the excess stain was wicked off, the grid was allowed to dry for at least 10 minutes before it was imaged on the electron microscope. Images were collected on a FEI Tecnai G2 transmission electron microscope (Hillsboro, Oreg.) at 80 kV with an AMT digital camera (Woburn, Mass.). Exosomes were characterized for marker expression using EVs were verified using Exo-Check Arrays (System Biosciences/SBI, Palo Alto, Calif., USA) for eight purported exosome markers.

### Animal Experiments

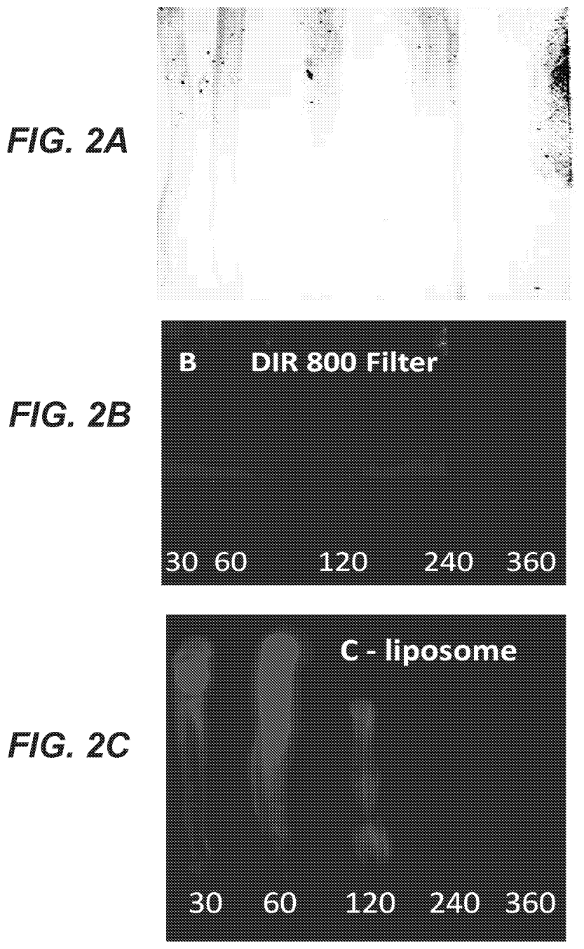

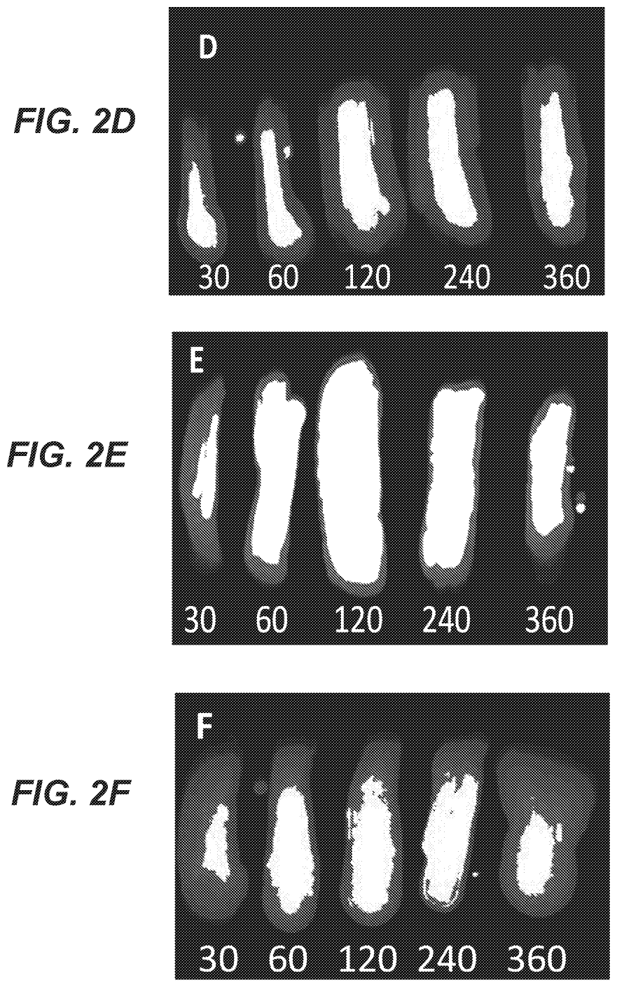

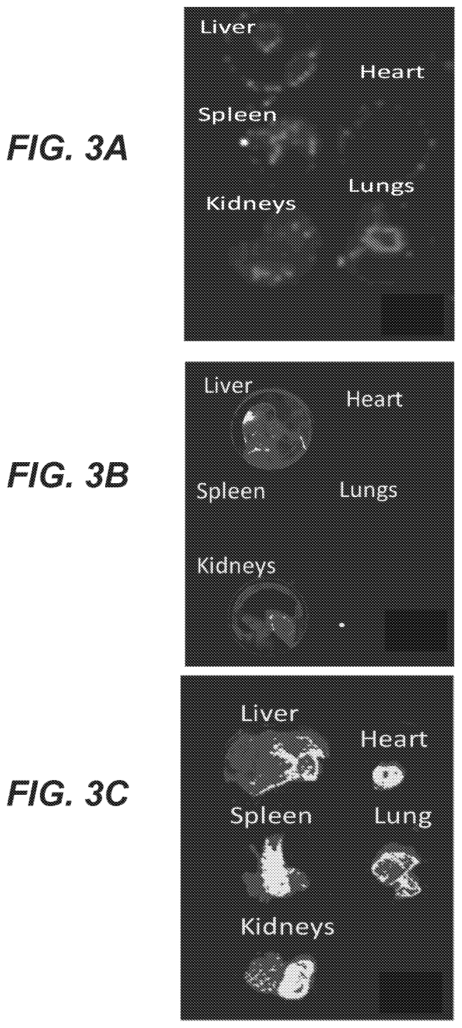

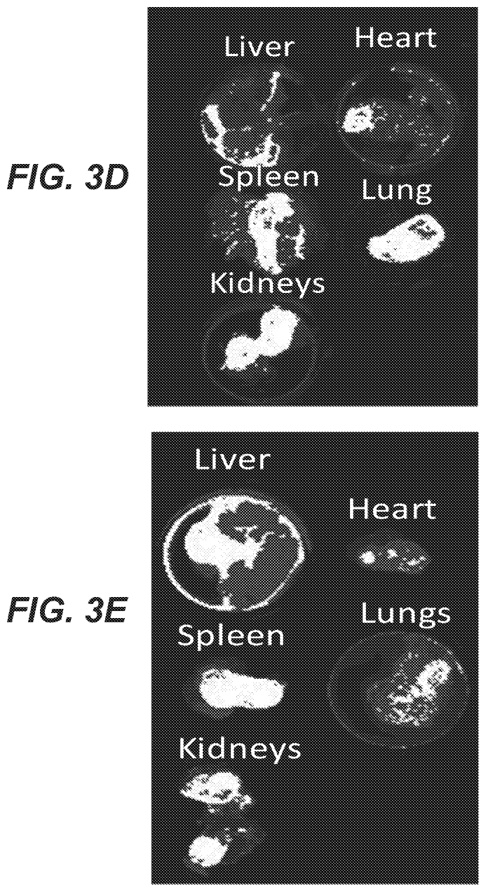

DIR labelled exosomes: Purified exosomes were labelled with DIR according to the protocol used in previous studies (T. Smyth, et al., Journal of Controlled Release 199:145-55 (2015)). Briefly, the dye is dissolved in ethanol at a concentration of 220 μg/mL. Five microliters of the dye are mixed with 220 μg/mL of exosomes (or control liposomes) in 100 ul PBS and incubated at room temperature for 1 h. Preparations were loaded on a sepharose column, and exosomes eluted with PBS to remove free dye. DIR-labelled milk exosomes were administered to female balb/c mice (6-10 weeks old; acquired from Jackson labs, Bar Harbor, Me.) via oral gavage at a dose of 40 mg exosome protein/kg in approximately 100 μl. For comparison, a single mouse was administered an equivalent dose of DIR-labelled liposomes (egg phosphatidylcholine, ≈100 nm). A single control mouse administered PBS was also included as a control for background fluorescence. Blood samples were collected at 30, 60, 120, 240 and 360 min from the sub-mandibular veins to evaluate both the extent and timing of absorption. A cotton swab was used to smear each blood sample onto a nitrocellulose membrane that was imaged on an Odyssey Imager (LI-COR Biosciences, Lincoln, Nebr.). Immediately after sacrifice, organs (liver spleen, kidney, heart, lung, blood via cardiac puncture) were harvested and placed in 12 or 24-well plates for imaging. All animal procedures were approved by the University of Colorado Institute for Animal Care and Use Committee in accordance with guidelines from the National Institutes of Health.
Dual labelled exosomes: Purified exosomes were labelled with SYTO 13 and DiD. Briefly, DiD was dissolved in DMSO to produce a 9.5 mM stock solution. SYTO 13 was supplied as a 5 mM stock solution in DMSO. Two hundred μg exosomal protein (as measured by BCA protein assay) were incubated with 50 μl of 50 μM SYTO 13 for 30 min at 37° C. Subsequently, 10 μl of 95 μM DiD were added to the exosomes and incubated for an additional 30 min at 37° C. Unincorporated dye was removed by passing the exosomes over a Sepharose CL-4B loaded spin column. Labeled exosomes were administered to female balb/c mice via oral gavage at a dose of 40 mg exosomal protein/kg. Blood was collected via cardiac puncture 4 hrs post-administration and allowed to clot overnight at 4° C. to obtain serum. Exosomes were obtained from serum by differential centrifugation. Briefly, serum was centrifuged at 20,000×g at 4° C. for 30 min to remove debris. Samples was then centrifuged at 110,000×g at 4° C. for 70 min using a SW28 rotor and pellets were washed in 1×PBS. Samples were centrifuged again at 110,000×g at 4° C. for 70 min and pellets were resuspended in 1×PBS. Exosome samples were mounted and imaged via laser scanning confocal microscopy using a Zeiss LSM780 microscope.
In vivo bovine IgG competition: Purified exosomes were labelled with DIR per the method described above, and bovine IgG purchased from Pierce Protein Biology (Thermo Fisher Rockford, Ill.) was administered via oral gavage. Bovine IgG was spin concentrated 3-fold using a 100 kDa Amicon Centrifugal filter (Millipore Burlington, Mass.). Exosomes (80 μg exosomal protein in approximately 100 μl) were administered by oral gavage immediately followed by bovine IgG at three doses (20 μg, 200 μg, 2000 μg). A similar experiment was conducted to determine the effect of a large oral dose of protein on exosome uptake. Accordingly, a protein that does not interact with FcRn (erythropoietin; 2000 μg) was administered immediately after exosomes. Blood samples were taken from the submandibular veins at 30, 60, 120, 180 minutes post-gavage. The mice were then sacrificed, and blood and organs were harvested. The blood was again smeared on a nitrocellulose membrane and the organs were imaged in 12- or 24-well plates on the Odyssey Imager.
Studies with Tumor-Bearing Mice: Because of the unexpectedly high levels of absorption and the signal saturation observed in our initial studies (FIGS. 2 and 3), the amount of DIR used to label exosomes was reduced by 10-fold in further experiments. These exosomes were used to assess tumor accumulation in immunocompetent tumor-bearing mice. Briefly, female balb/c mice were inoculated in the flank with murine colon carcinoma cells (CT26), and tumors were allowed to grow to approx. 100 mm³. Triplicate mice were administered DIR-labelled milk exosomes, and tissues were harvested after 4 h. To assess the ability to target exosomes with ligands, iRGD-cholesterol was incorporated into exosomes at a concentration of 0.1% using the same procedure described above for DIR loading. The term “iRGD” refers to a family of related peptide ligands that bind specifically to the αv β3/β5 integrin on tumor vasculature and promote transcytosis across tumor vasculature. After translocation, the iRGD ligand is cleaved by an endogenous protease to yield a peptide that serves as a ligand for the neuropilin-1 receptor to promote uptake by cancer cells. Previous studies have shown that cyclic peptides can survive in simulated gastric fluid conditions. Accordingly, another set of triplicate mice were administered DIR-labelled, iRGD-targeted exosomes. To allow comparison with IV administration, single mice were IV injected with either free DIR or DIR-labelled milk exosomes. To simulate comparable dosing via the two routes of administration, the efficiency of oral absorption was estimated at 10%, and thus mice receiving IV injection received only one-tenth of the DIR dose that was administered via oral gavage. After 4 h, blood samples were collected and imaged as described above. In the experiments involving iRGD-containing exosomes, tissues were homogenized in homogenization buffer (250 mM sucrose, 1 mM EDTA, 10 mM Tris HCL pH 7.2 and protease inhibitor cocktail from Pierce Protein Biology Thermo Fisher, Rockford, Ill.) 4 h after oral gavage, and the fluorescent signal from accumulated DIR-labelled exosomes was measured with an Odyssey Imager. Values for DIR fluorescence (RLU) were standardized against the protein levels in each tissue homogenate. Exosome protein levels were determined by BCA assay (Pierce Thermo Fisher Rockford, Ill.). Other methods for protein quantitation were tested including a Bradford (Biorad Hercules, Calif.), phase extraction, and a lysis protocol. Protein levels varied dramatically with each assay, hence it is important to be cognizant of which assay is chosen when quantifying exosomal protein. The BCA assay was chosen to be consistent with previous studies by Gupta and colleagues with respect to measurements and dosing (R. Munagala, et al., Cancer Lett 371(1):48-61 (2016); A. K. Agrawal, et al., Nanomed-Nanotechnol 13(5):1627-36 (2017).
Western Blot: Purified milk exosomes were precipitated with ExoQuick (System BioSciences, Palo Alto, Calif.); one batch was untreated (XOQ ppt), and the other batch was treated with 0.2 M glycine, pH 2.0 to strip antibodies. Glycine-treated exosomes (XOs strip) and eluted antibodies (eluted Abs) were separated by centrifugation through a 300 kDa cutoff membrane. Samples were electrophoresed, transferred to nitrocellulose (Western blot), and probed with goat anti-bovine IgG (H&L chain)-HRP, 1:2000 dilution (ThermoFisher Scientific). The blot was developed with chemiluminescence, and imaged with a FluroChem Q Imager III device (ProteinSimple, Santa Clara Calif.). Bovine γ-globulin (Bio-Rad Life Sciences Research) was included as a positive control, and “unbound” antibodies came from the ExoQuick supernatant.

### Example 1

### Characterization of Exosomes from Cow Milk

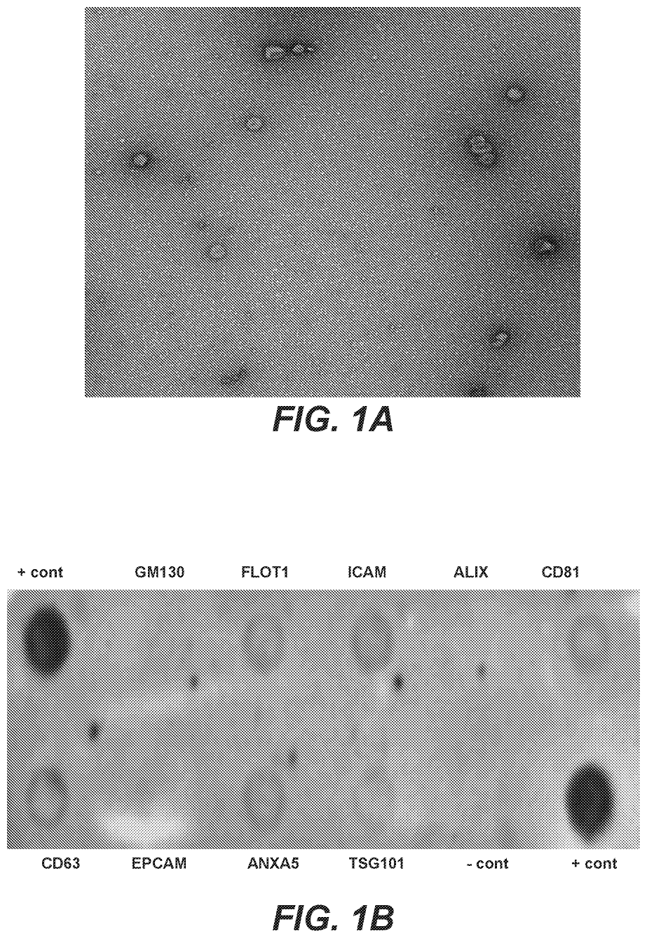

To characterize exosomes extracted from cow milk, fresh milk was acquired from a local dairy and harvested exosomes according to previously described protocols (Agrawal et al. (Nanomed-Nanotechnol 13(5):1627-36 (2017)). As shown in FIG. 1A, vesicles were isolated having sizes consistent with exosomes. As observed in previous work with exosomes, dehydration of samples for electron microscopy causes some clumping and yields slightly smaller particle sizes as compared to the hydrated diameters measured by dynamic light scattering (127 nm). Consistent with other exosome preparations, the size distribution is quite uniform, and particles possess a negative charge (−15.2 mV). The variability of exosomes prepared from milk obtained from cows at different stages of lactation (early, mid, late) was also examined. The size and zeta potential of exosomes harvested from four different cows were very consistent, and hydrated diameters ranged from 127 to 140 nm, and zeta potentials ranged from −15.2 to −7.6 mV.

Exosome marker characterization (using an Exo-Check Array) showed that bovine exosomes have markers consistent with other exosomes (FIG. 1B). ALIX was not apparent in the preparations, consistent with the protein's presence in exosomes from colostrum, but not mature milk. EPCAM was also not found identified on bovine milk exosomes in the literature.

### Example 2

### Characterization of Absorption of Gastrointestinal Tract

The extent to which exosomes are absorbed from the gastrointestinal tract was assessed. To track uptake, an infrared dye, DIR, was utilized that has been previously validated/optimized for tracking intravenously-administered exosomes. DIR-labelled milk exosomes were administered to mice via oral gavage at a dose of 40 mg exosome protein/kg. For comparison, a single mouse was administered an equivalent dose of DIR-labelled liposomes (egg phosphatidylcholine, ≈100 nm), and a single control mouse administered PBS was also included as a control for background fluorescence. As shown in FIGS. 2A-2F, blood smears from mice administered PBS exhibited undetectable levels of background infrared signal. Blood from mice administered DIR-labelled liposomes had a low infrared signal at 30 min, which increased at 60 min, diminished by 120 min, and was undetectable at later time points. In sharp contrast to the results with liposomes, the infrared signal of blood from mice administered exosomes saturated our detector, even on the lowest sensitivity setting. The area of signal saturation increased after 30 min but remained relatively constant (almost completely saturated) from 60-240 min, and then diminished slightly after 360 min in all mice. These results clearly demonstrate that molecules loaded into milk exosomes (in contrast to liposomes) are readily absorbed. Additionally, the timing of absorption is surprisingly rapid, and sufficient quantities of dye are in the circulation after only 30 min. Furthermore, high levels of dye are sustained for at least 6 h. This initial experiment demonstrates that milk exosomes can facilitate the transfer of significant quantities of molecular cargo into the blood after oral administration. Moreover, the fact that blood from mice administered DIR-labelled liposomes had markedly lower absorption suggests that the mechanism of absorption is not simply due to random particle uptake.

### Example 3

### Uptake into Organs

The mice described in Example 2 were sacrificed after 6 h, at which time the liver, spleen, kidney, heart and lungs were harvested and placed in 24-well plates for imaging. As shown in FIGS. 3A-3E, background levels of IR signal were barely detectable in organs from mice administered PBS. The signal from mice administered DIR-labelled liposomes was clearly evident in the kidneys and liver. In contrast, organs from mice administered DIR-labelled milk exosomes displayed large regions in which the IR detector was saturated. Consistent with the high levels of absorption into the blood seen in FIGS. 2A-2F, large amounts of dye accumulate in all organs 6 h after oral administration of milk exosomes. It is important to point out that blood levels of dye are still high at this time point, and thus tissue accumulation will likely be greater at longer times. These data are consistent with previous studies showing that milk exosomes facilitate the transfer of cargo into the blood, which ultimately accumulates in tissues.

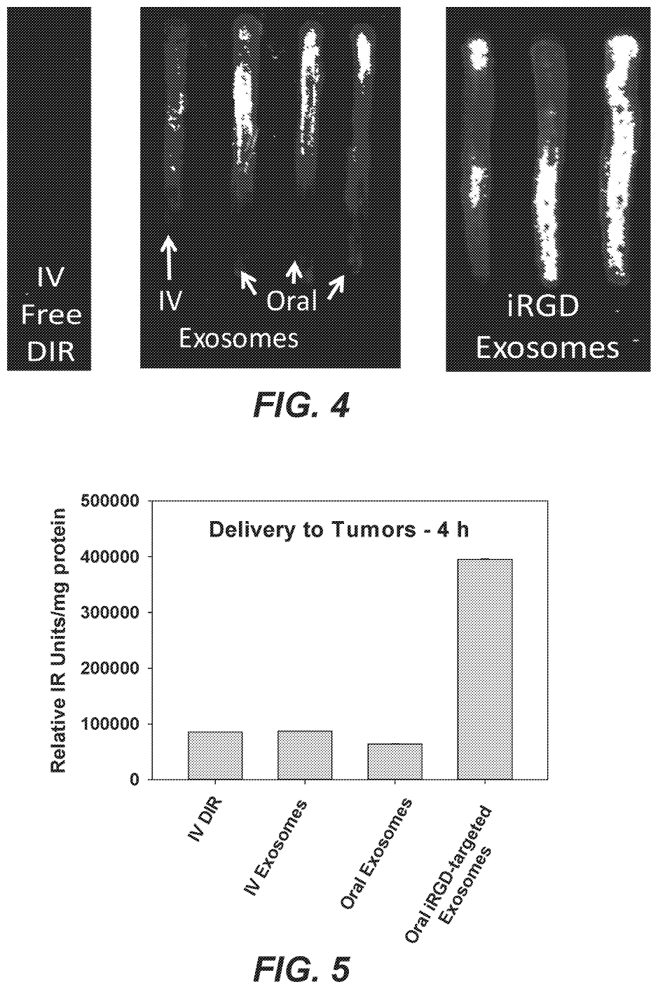

Because of the unexpectedly high levels of absorption and the signal saturation observed above, the amount of DIR used to label exosomes was reduced by ten-fold in future experiments. These exosomes were then used to assess tumor accumulation in immunocompetent tumor-bearing mice after 4 h. To more effectively compare fluorescent signals from blood achieved after IV versus oral administration, IV doses were reduced an additional ten-fold. As shown in FIG. 4, no signal was seen in blood from mice administered free DIR by IV injection, supporting that free dye is cleared from the blood within 4 h. Blood from mice administered DIR-labelled exosomes via IV injection had a strong IR signal that was just below the saturation limit of the detector. However, blood from mice dosed via oral gavage had a stronger signal that exceeded the saturation limit (FIG. 4). Furthermore, the incorporation of the iRGD ligand into exosomes did not substantially alter absorption. Although these data are not quantitative, the fact that blood levels were higher after oral gavage, as compared to that from mice administered a ten-fold lower dose via IV injection, supports that oral bioavailability is greater than 10% under these conditions.

### Example 4

### Accumulation in Tumors

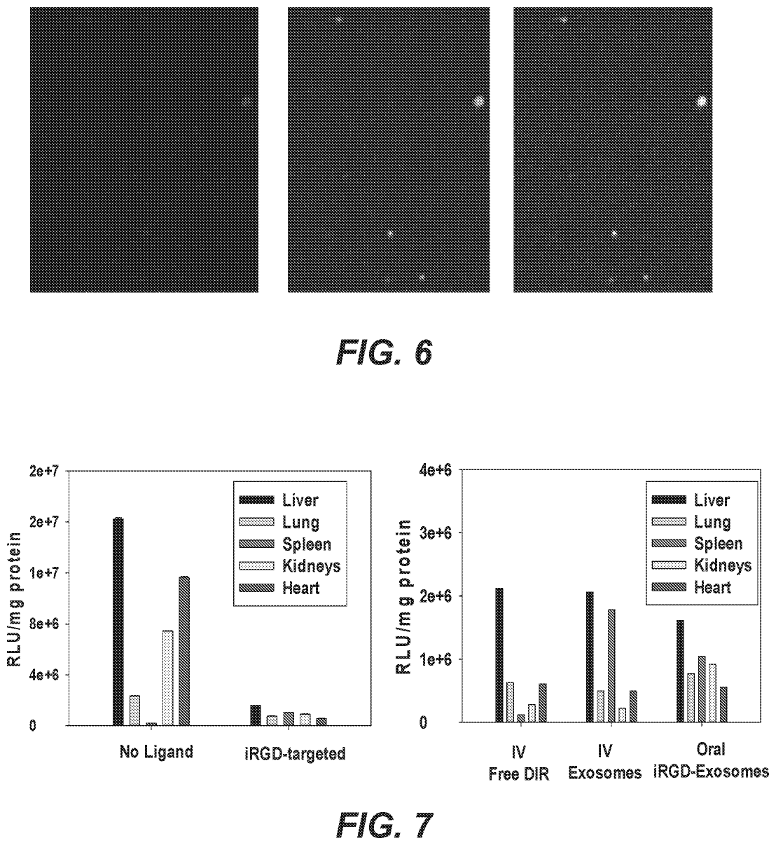

To assess tumor accumulation, tumors were harvested from mice after 4 h. To obtain semi-quantitative results, tumors were homogenized and the relative fluorescence units in the resulting suspension were quantified with the Odyssey imager and standardized against the protein content. FIG. 5 depicts the relative amounts of dye in tumors from mice receiving oral gavage as compared to mice dosed via intravenous injection. The results indicate that comparable levels of tumor accumulation were achieved via oral and intravenous administration (10% of the oral dose), and that the iRGD ligand significantly increased tumor accumulation. The fact that a ligand alters accumulation after oral absorption evidences that the dye remains associated with the ligand, potentially in an intact exosome. An additional experiment was conducted wherein both the membrane and RNA encapsulated within milk exosomes were fluorescently-labelled, and these double-labelled exosomes were orally administered to three mice as described above. Blood from the mice was collected after 4 h, and exosomes were isolated from the pooled blood samples and analyzed by confocal microscopy. As shown in FIG. 6, some exosomes isolated from the blood of mice possessed both fluorescent labels, demonstrating that milk exosomes can be absorbed from the gastrointestinal tract as intact particles. This is consistent with the greater tumor accumulation observed with iRGD-containing exosomes (FIG. 5). It is important to note that previous studies have shown that particles can be altered during transcytosis, even if they remain intact.

In addition to the tumor, other organs were harvested after 4 h, homogenized, and analyzed for IR signal. As is commonly observed in drug delivery, liver accumulation was the highest of all the organs, consistent with clearance (FIG. 7). Organ accumulation of iRGD-containing exosomes was dramatically reduced despite enhanced tumor distribution and comparable absorption from the gastrointestinal tract. This result is very encouraging in terms of limiting off-target effects, it demonstrates that iRGD-containing exosomes are excreted more efficiently and/or accumulate at a different site.

### Example 5

### Interactions with FcRn Receptor

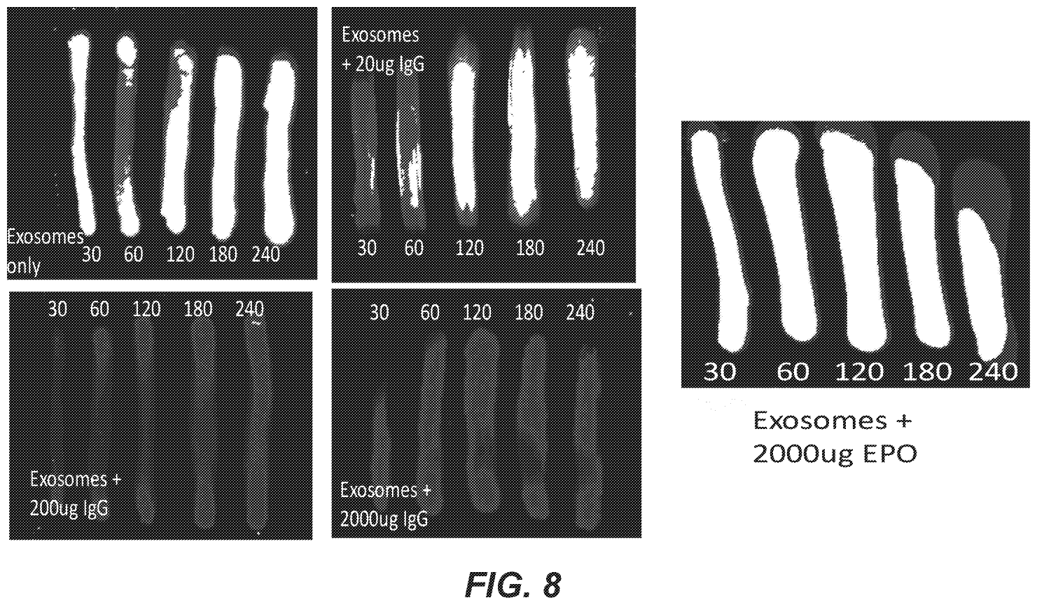

The results presented above are consistent with reports claiming that milk exosomes are absorbed from the gastrointestinal tract. These findings can be viewed more broadly in terms of the ability of exosomes (i.e., lipid nanoparticles) to cross other cell barriers (e.g., the blood-brain barrier). This phenomenon has gained a tremendous amount of interest, but no mechanistic explanation for this effect has been proposed. However, previous studies have demonstrated the ability of FcRn to facilitate transport of Fc-targeted particles across the gastrointestinal tract (H. M. Patel, et al., Febs Lett 234(2):321-25 (1988); E. M. Pridgen, et al., Sci Transl Med 5(213) (2013)). Furthermore, it is important to recognize that FcRn is expressed in the vascular endothelium as well as the gastrointestinal tract throughout life in humans. Because both mouse and human FcRn bind bovine IgG, it was tested whether interactions with this receptor play a critical role in the ability of milk exosomes to be absorbed after oral administration. Mice were co-administered a constant amount of DIR-labeled exosomes with varying amounts of free bovine IgG. It follows that the free IgG should compete with exosomes for FcRn in the gastrointestinal tract, and should thereby reduce absorption. As before, blood samples were collected at different timepoints and imaged, and FIG. 8 clearly shows that the IR signal intensity diminishes at higher doses of co-administered IgG. To assess the potential for this effect to be simply due to the presence of increased protein in the gut, a parallel experiment was conducted in which erythropoietin was co-administered with exosomes, but no effect of this protein was observed (FIG. 8). Virtually identical effects on organ accumulation were observed. These studies demonstrate that co-administration of bovine IgG reduces uptake, and that this effect is not generally observed with other proteins. Taken together, these data strongly support that FcRn is involved in absorption of milk exosomes from the gastrointestinal tract. However, the significant absorption of exosomes observed despite swamping amounts of co-administered IgG also indicates that other factors contribute to gastrointestinal uptake.

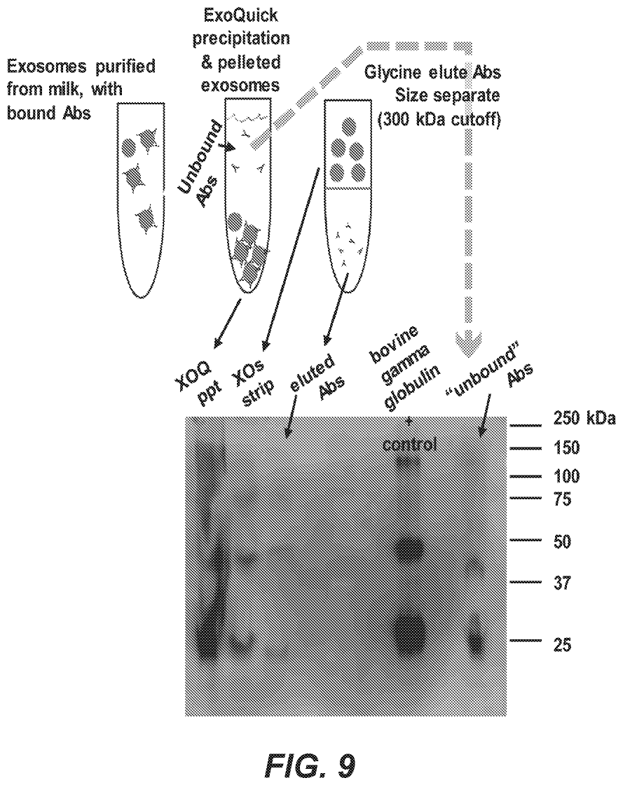

It seems evident that utilization of the gut epithelial FcRn would require Fc ligand on milk exosomes; however, this has not been specifically explored. Other reports (Samuel, et al., Sci Rep-Uk 7 (2017); Reinhardt, et al., J Proteomics 75(5) (2012) 1486-1492; Reinhardt, et al., J Proteomics 82 (2013) 141-154; Yang, et al., Food Res Int 92 (2017) 17-25; Graner, et al., Proc SPIE 8723, Sensign Technologies for Global Health Military Medicine, and Environmental Monitoring!!! (2013)) have identified immunoglobulin (Ig) components via proteomics of cow milk exosomes. Following a similar protocol as described previously (Graner, et al., Proc SPIE 8723, Sensign Technologies for Global Health Military Medicine, and Environmental Monitoring (2013)), the presence of both tightly-bound and elutable bovine IgG in/on cow milk exosomes was detected (FIG. 9). These results demonstrate that there are substantial amounts of bovine IgG in/on cow milk exosomes, and the majority of it remains associated with the exosomes following the glycine stripping. Densitometry determinations indicate that cow milk exosomes have 1-2 μg IgG per 50 μg of exosome protein. Although this may seem like a high percentage, cow milk contains 300-600 μg/ml of IgG, therefore the exosome IgG concentrations are reasonable.

Although oral dosage forms are clearly preferable for patients, many drugs require parenteral administration due to poor oral bioavailability. The reasons for poor oral bioavailability can be multiple, but typically involve low aqueous solubility, instability under the conditions in the gastrointestinal tract, low absorption through the gut epithelium, and/or the first-pass effect in the liver. The use of particulate delivery systems for oral delivery has the potential to circumvent many of these issues, but uptake of particles from the gastrointestinal tract is the predominant barrier. Indeed, previous work with lipid-based formulations and emulsions have been used to improve the solubility of lipophilic drugs and enhance permeability of the intestinal endothelium. Successful oral delivery could be achieved by using particles that release drug into the intestinal epithelial cells and rely on subsequent diffusion to access the systemic circulation. This scenario would appear more feasible, albeit potentially less efficient, because it does not require uptake of intact delivery systems from the gastrointestinal tract and subsequent transport across the epithelium.

It is well established that exosomes play a role in many biological processes and possess the ability to transfer molecules among cells. Previous research has attempted to harvest exosomes from cells in culture for use as delivery vehicles. Although some promising results have been reported, the inability to produce and harvest exosomes on a commercial scale has always been a concern associated with exosome-mediated delivery. In this regard, the isolation of small quantities of exosomes needed for in vitro experiments is labor-intensive, but feasible. However, in vivo experiments and potential clinical studies require that large amounts of exosomes be harvested from a readily-available source. It is now recognized that all bodily fluids (e.g., blood, urine, lymph, milk) contain exosomes that are secreted by every cell type. Exosomes from mother's milk have evolved to resist conditions encountered in the stomach to transfer molecules to the baby. Studies have shown that exosomes from cow milk are capable of withstanding simulated gastric conditions, maintaining and transferring their contents from the gastrointestinal tract into the blood. With particular relevance to the clinical application of this approach, it is important to point out that a human study documented that functional miRNAs from cow milk are absorbed into the circulation (Baier, et al., J Nutr 144(10):1495-1500 (2014)). Furthermore, previous studies in mice have demonstrated that exosomes can be isolated from cow milk and used to transfer molecules across the gastrointestinal epithelium. The results of this experiment are consistent with these previous studies and demonstrate that significant quantities of exosomes are absorbed from the gut within 30 min, and blood levels remain high for at least 6 h in mice (FIGS. 2A-2F). In addition, significant levels of orally-administered exosomes were observed in all tissues after 6 h (FIGS. 3A-3E).

These experiments in tumor-bearing mice also demonstrate that ligands can be incorporated into orally-delivered exosomes to enhance accumulation in tumors (FIG. 5). In addition to enhanced tumor accumulation, a reduced accumulation in other organs was observed as compared to exosomes lacking the iRGD ligand (FIG. 7). The ability of the ligand to alter the distribution profile indicates that the ligand remains associated with the exosome throughout absorption and distribution via the systemic circulation. This supports that milk exosomes can be absorbed as intact particles from the gastrointestinal tract, and previous studies have suggested that some level of repackaging may occur during passage through the intestinal epithelium. These results from the double-label experiment demonstrate that at least some exosomes are absorbed as intact particles (FIG. 6).

Although previous studies have utilized milk exosomes for oral delivery, a plausible mechanism by which exosomes (especially intact exosomes) could be absorbed from the gastrointestinal tract has been lacking. Considering the ability of FcRn to salvage IgG and transport it from the gastrointestinal tract into the blood, researchers have attempted to exploit this natural mechanism to transcytose particulate delivery systems across the gut after oral administration. Early studies conducted with liposomes demonstrated that IgG could be used as a ligand to facilitate uptake from the gut into the circulation (Patel, and Wild, Febs Lett 234(2) (1988) 321-25). More recent studies have utilized a similar approach with Fc-targeted PLA nanoparticles in an attempt to develop an orally-administered form of insulin (Alexis, Sci Transl Med 5(213) (2013)). Like the earlier studies with liposomes, the researchers demonstrated significant oral bioavailability with Fc-targeted particles, and achieved very high mean absorption efficiencies of 13.7±1.3%/hr. In contrast, non-targeted particles exhibited an oral absorption efficiency of 1.2±0.2%/hr. As noted above, the inventors' previous proteomic work has documented the presence of antibody fragments on purified exosomes, supporting a role in uptake via FcRn. These experiments further demonstrate that co-administration of bovine IgG substantially reduces the levels of exosomes absorbed into the blood is consistent with this hypothesis (FIG. 8). The fact that co-administration of another protein (i.e., erythropoietin) did not affect absorption indicates that this effect is specific to IgG, consistent with uptake involving FcRn.

In considering the absorption of bovine exosomes across the intestinal epithelium of mice or humans, it is important to recognize that studies quantifying the relative binding affinities of bovine IgG for murine and human FcRn have documented significant cross-reactivity among these species. More specifically, the affinity of bovine IgG for the mouse receptor is comparable to that of murine IgG. With regards to uptake in humans, the relative binding of bovine IgG for the human receptor is approximately 28% of that for human IgG. Considering that lactating cows have evolved mechanisms to pass materials through the intestinal epithelium of their calves, it is conceivable that the cross-reactivity with human FcRn could permit similar transport, albeit with reduced efficiency. These reports are consistent with a role for FcRn in the absorption of milk exosomes observed in both humans and mouse models, presumably via transcytosis, and additional factors that work in collaboration with FcRn also may be involved in transport across the intestinal epithelium, resulting in highly-efficient transport of the nanoparticles of this disclosure across epithelia.

It is worth considering differences between exosome-mediated delivery and conventional nanoparticles in terms of drug release and tumor delivery. As shown in FIG. 4, high blood levels of dye are observed 4 h after exosome-mediated delivery regardless of the route of administration (e.g., oral or intravenous). In contrast, intravenous administration of free dye resulted in undetectable blood levels under the same conditions, indicating that dye remains sequestered within exosomes after gastrointestinal uptake. This agrees with the findings in FIG. 6 demonstrating that exosomes are transported across the gastrointestinal epithelium as intact particles that are dissociated after internalization by the recipient cell. This scenario is consistent with the biological role of exosomes in the intercellular transfer of microRNAs that would likely not survive exposure to the extracellular environment. Taken together, these data indicate that dye release from circulating exosomes is minimal, and that drugs encapsulated within exosomes may be most effective against intracellular targets. However, the data in FIGS. 5 and 7 suggest that molecules present on the exterior surface of exosomes can access cell receptors (i.e., the mechanism of iRGD targeting involves binding to the αv β3/β5 integrin on tumor vasculature), indicating that it is possible to achieve both targeting via surface moieties and efficient intracellular delivery after accessing the circulation. This will prove especially advantageous for reducing the systemic distribution and off-target effects of chemotherapy if efficacious tumor targeting can be achieved. These results showing increased tumor accumulation and reduced organ accumulation by employing iRGD-mediated targeting demonstrate that.

### Example 6

### Loading Nanoparticles with Therapeutic Agent

Exosomes isolated from cow's milk as described in Example 1 were loaded with SN38 (an anticancer drug that is an active metabolite of irinotecan, a topoisomerase I inhibitor) and characterized by ultra-performance liquid chromatography (UPLC).

Briefly, 45 μl exosomes were incubated with 5 μl 100% EtOH for 20 min at room temperature. 30 μg of SN38 in 10 μl of n-methylpyrrolidone (NMP) and 90 μl 0.01M phosphate buffer, pH 5, were added to the exosomes. The composition was mixed and incubated 20 min at room temperature.

The exosomes were separated from free drug on a sepharose column conditioned with 0.01M phosphate buffer, pH 5. After the initial flow through to load the exosomes onto the sepharose column, the remaining exosomes were eluted with four washes of the column with 150 μl of 0.01M phosphate buffer, pH 5. By the fourth wash, 95% of the exosomes were eluted from the column). The volume of the phosphate wash buffer was then increased to 300 μl for two additional washes of the column.

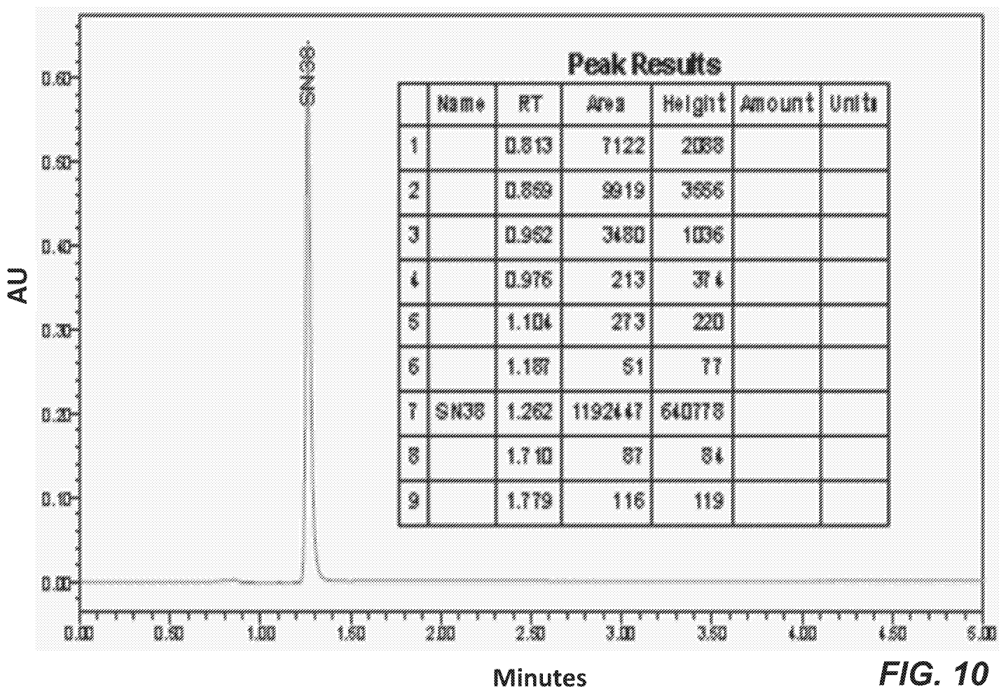

An equal volume of acetonitrile was added to each wash solution eluted from the column, mixed well and incubated 10 min at room temperature. The acetonitrile was dried off under a nitrogen stream, and the exosomes loaded with SN38 were suspended in 700 μl of 0.01M phosphate buffer, pH 5, and quantified by UPLC. FIG. 10 shows a chromatogram of the eluate from the third wash of the column, demonstrating that the inventors can quantify SN38 by UPLC.

Quantification of drug loading was complicated by protein contamination in the exosome preparation, and the quantification included protein-bound drug that was not loaded into exosomes. Thus, protein present in the exosome preparation can present problems for drug loading into the exosomes in that the protein (likely to be BSA) binds to the therapeutic agent being loaded into the exosome nanoparticles. Additionally, BSA-bound drug may not be absorbed from the gut because albumin is also known to bind to FcRn receptors.

### Example 7

### Loading Nanoparticles Isolated Via Tangential Flow Filtration with SN38

Exosomes from cow's milk were isolated by tangential flow filtration. The exosomal protein concentration was determined by BCA assay, and the total weight of exosomes was calculated by doubling the protein value (from BCA) to account for an approximate 50% content of protein in biological membranes (Smyth T. J., Redzic J. S., Graner M. W., Anchordoquy T. J. 2014. Examination of the specificity of tumor cell derived exosomes with tumor cells in vitro. Biochmica et Biophysica Acta, 1838:2954-65).

Tangential flow filtration was used to isolate exosomes from bovine milk over the course of 4 batches. A 2 L volume of milk was centrifuged at 13000×g for 30 min at 4° C. The fat-clarified milk was poured over cheesecloth and subsequently centrifuged at 100,000×g for 1 hr at 4° C. The supernatant was transferred to new tubes, avoiding the slush portion at the bottom of the tubes. This supernatant was then centrifuged at 135,000×g for 90 min at 4° C. The supernatant was discarded and exosomal pellets were resuspended in 20 ml 1×PBS. The resuspended pellets were pooled and added to the container for the tangential flow filtration device which had 3 manifolded membranes, 2 at 500K and 1 at 1000K (Pall Minimate). Table 1 summarizes the TFF process for each batch prepared from the initial 2 L volume.

TABLE 1

| | | Volume of | | | | | |
|---|---|---|---|---|---|---|---|
| | Volume of fat- | exosomes | RPM | Wash 1 | Wash 2 | Wash 3 | Wash 4 |
| Prep | clarified milk for | for TFF | for | initial | initial | initial | initial |
| Identification | ultracentrifugation | ultrafiltration | TFF | volume | volume | volume | volume |
| 8-1 | 180 ml | 120 ml | 350 | 200 ml | 200 ml | 200 ml | |
| 8-2 | 360 ml | 240 ml | 250 | 400 ml | 300 ml | 300 ml | 300 ml |
| 8-3 | 540 ml | 380 ml | 200 | 400 ml | 200 ml | 200 ml | |
| 8-4 | 505 ml | 250 ml | 200 | 400 ml | 200 ml | 200 ml | |

Several retentate samples were taken over the course of the TFF process to measure protein concentration and particle size and concentration. Permeate samples were also taken, but particles were only measured for the TFF process for preparation 8-4. As the TFF process results in very concentrated preparations of exosomes, samples are diluted to achieve 6 mg protein (12 mg exosomes) per milliliter (Munagala R, Aqil F, Jeyabalan J, Gupta R. C. 2016. Bovine milk-derived exosomes for drug delivery. Cancer Letter, 371:48-61) prior to drug loading.

SN38 was dissolved in N-methyl pyrrolidone (NMP) and added to exosomes at a 9:1 weight ratio of exosomes to drug. To assess the effect of using a buffer in the loading procedure, we compared drug loading in 10 mM phosphate buffer, pH 5 to that without the use of buffer. When buffer was added, the volume was adjusted to achieve a maximum NMP concentration of 10%.

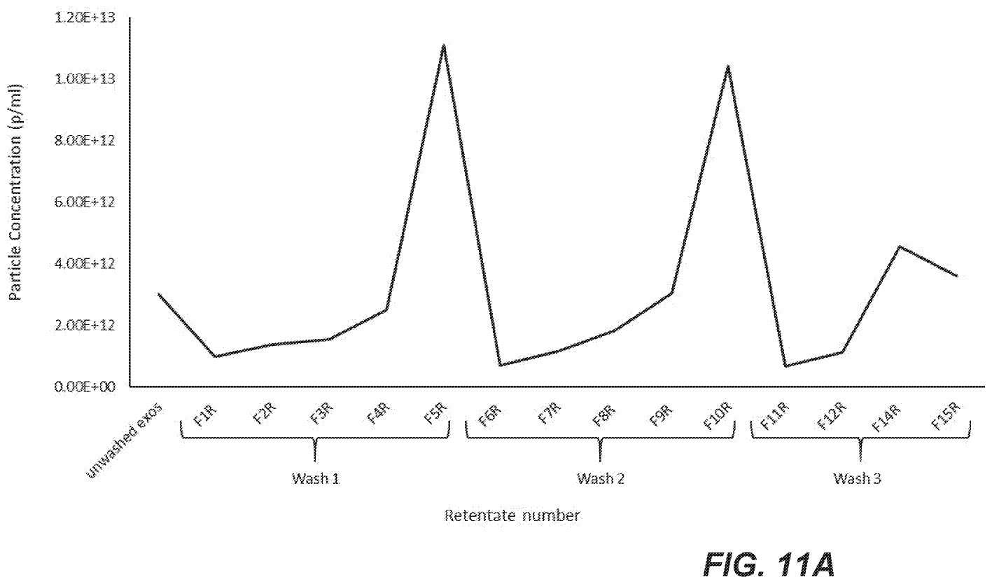

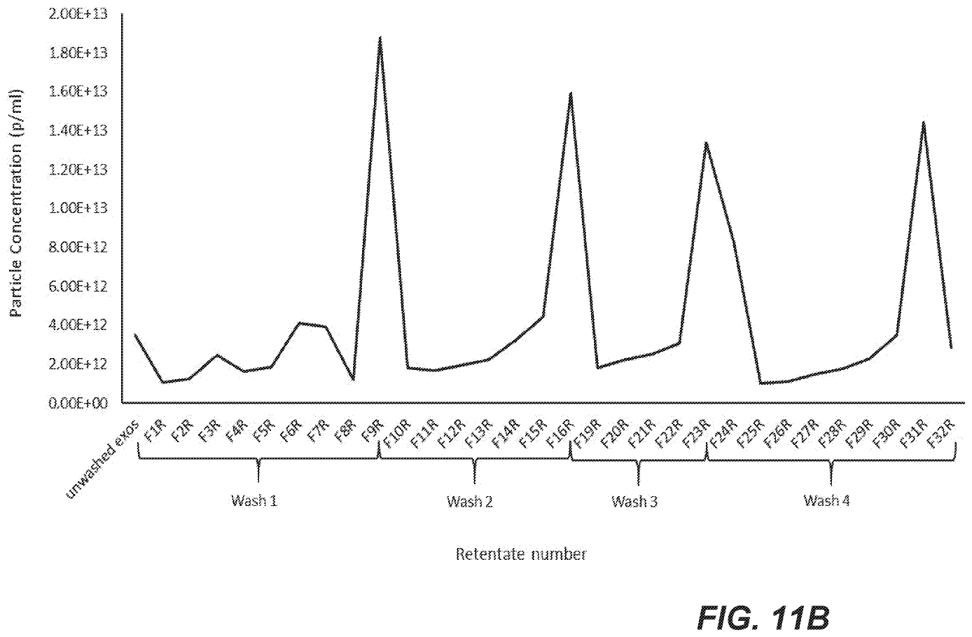

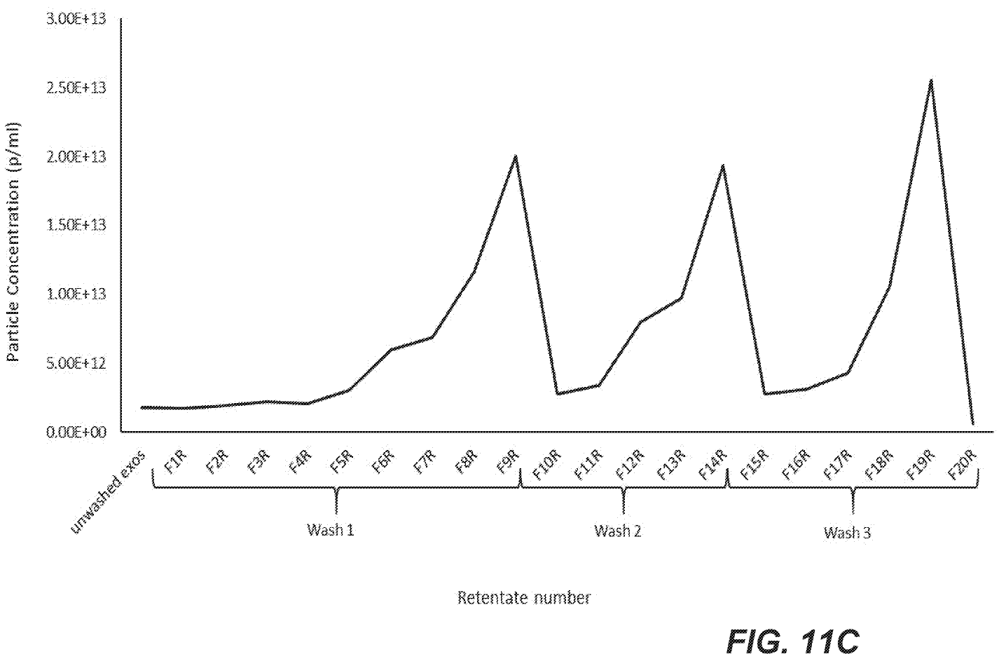

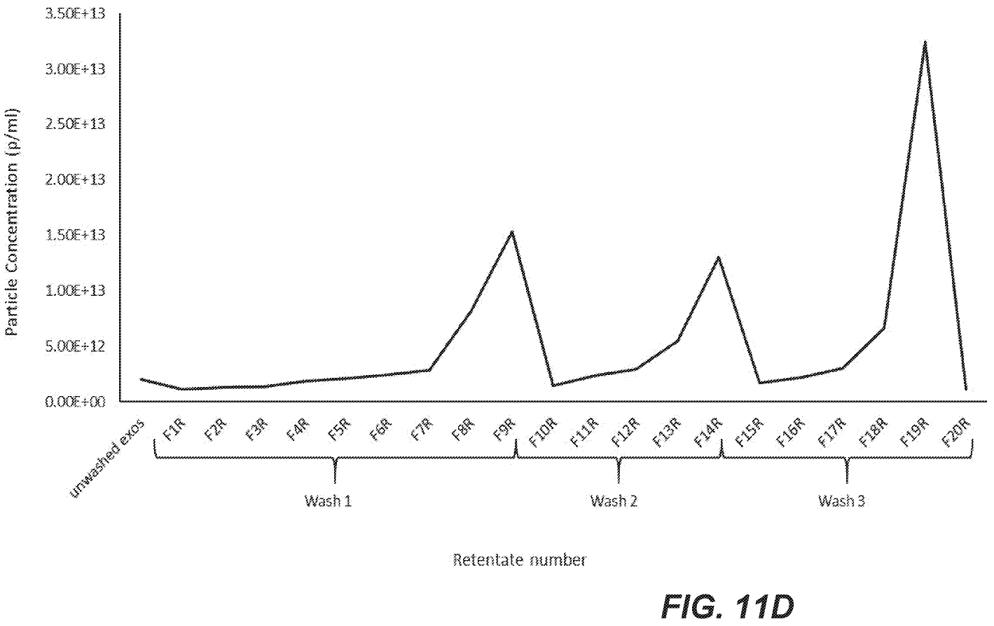

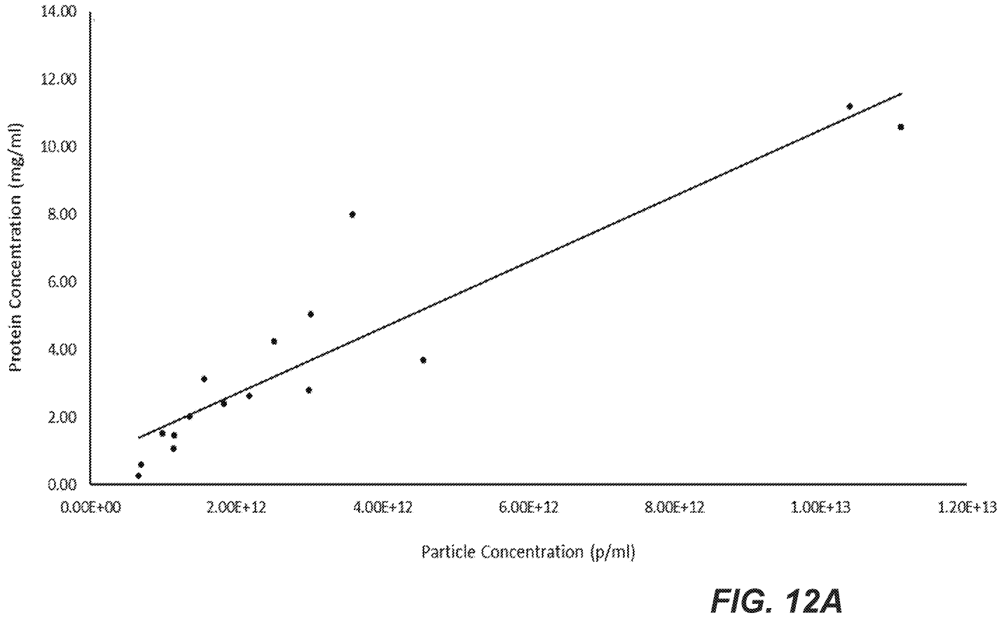

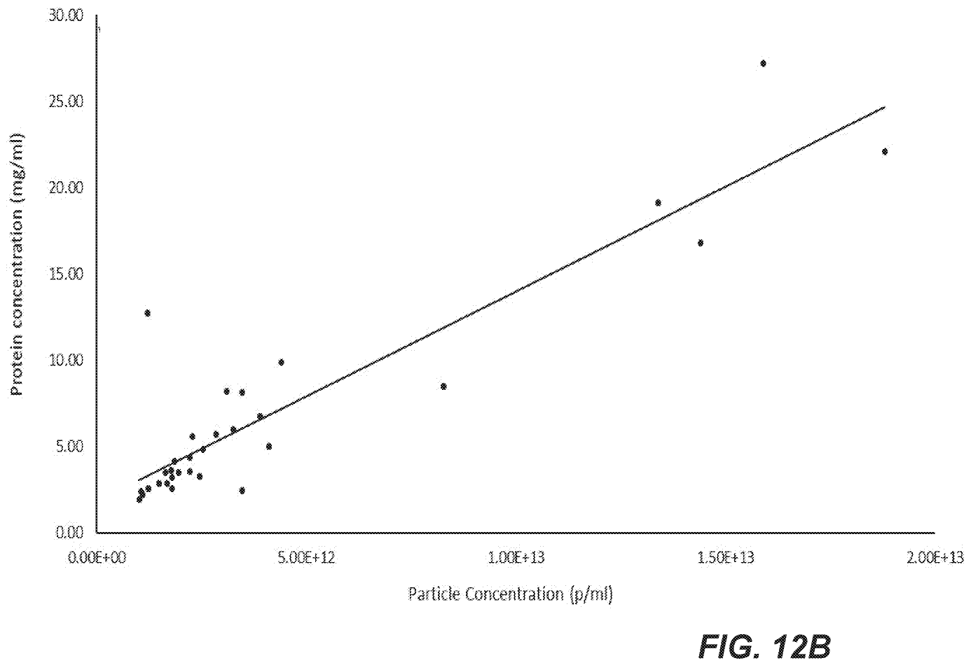

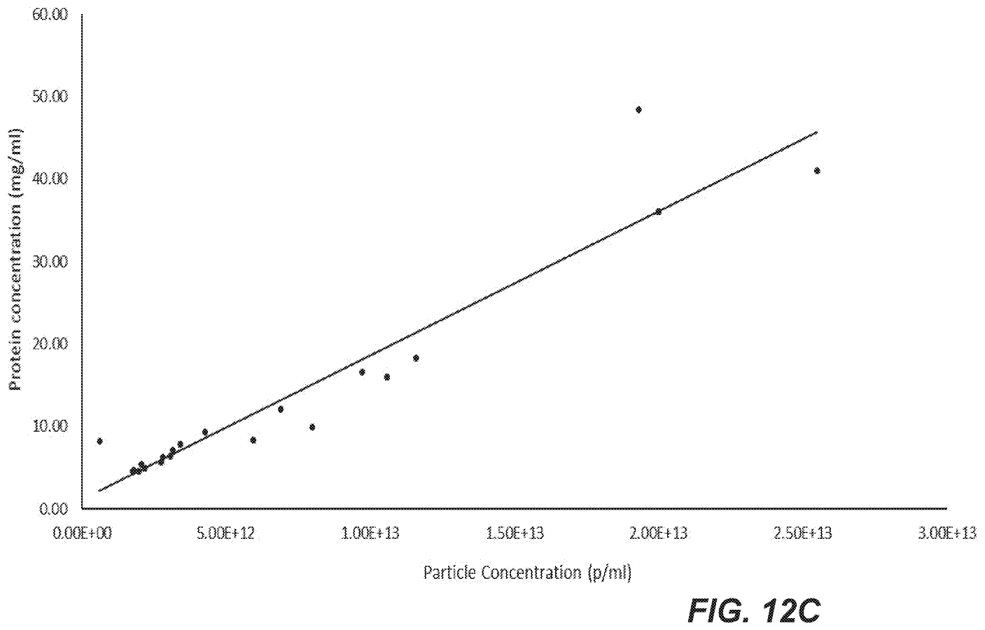

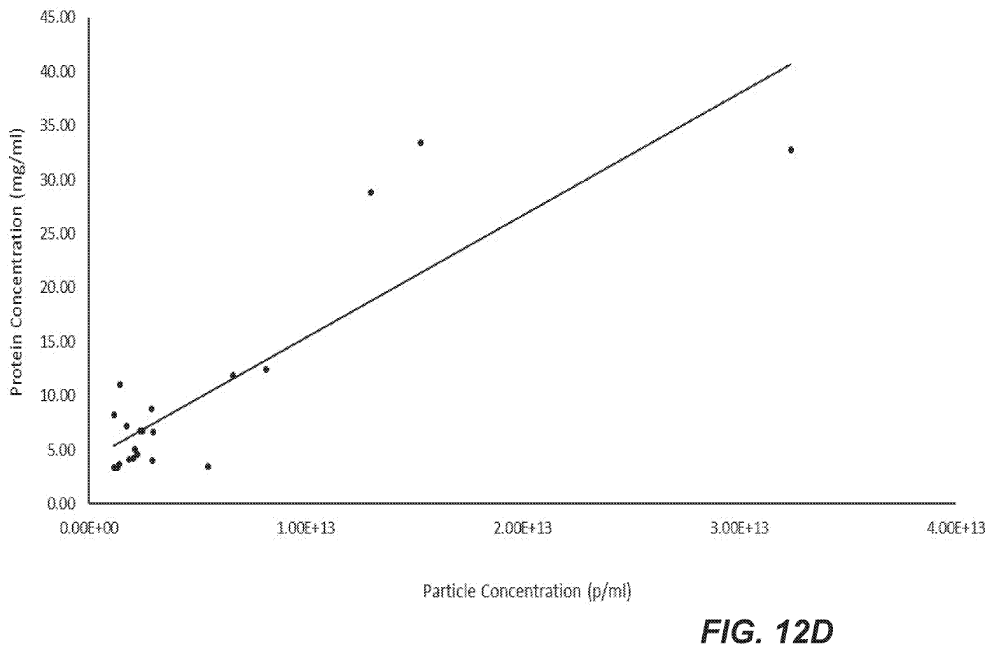

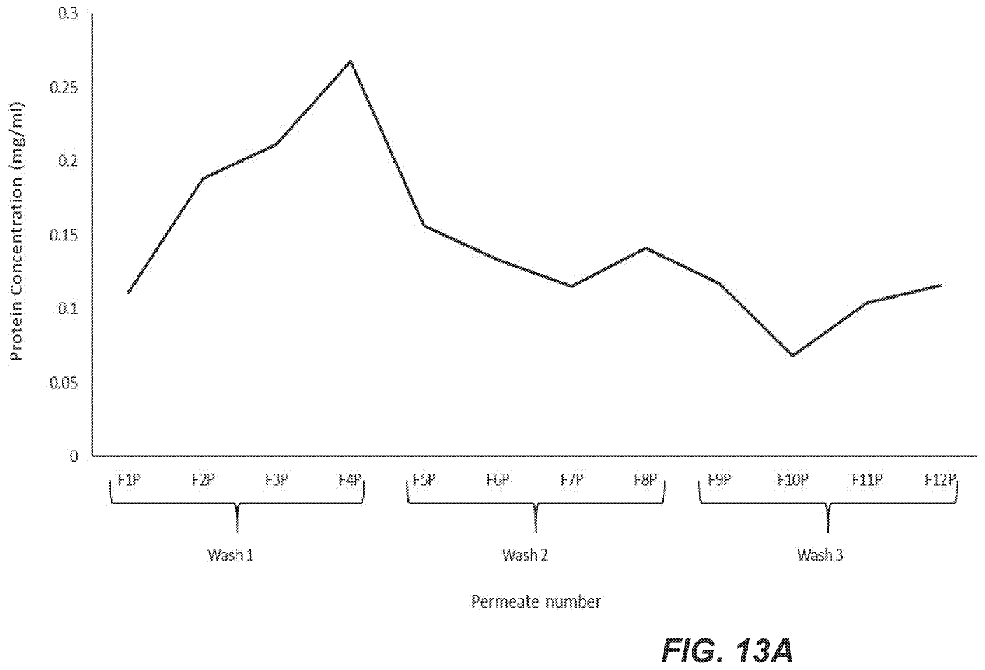

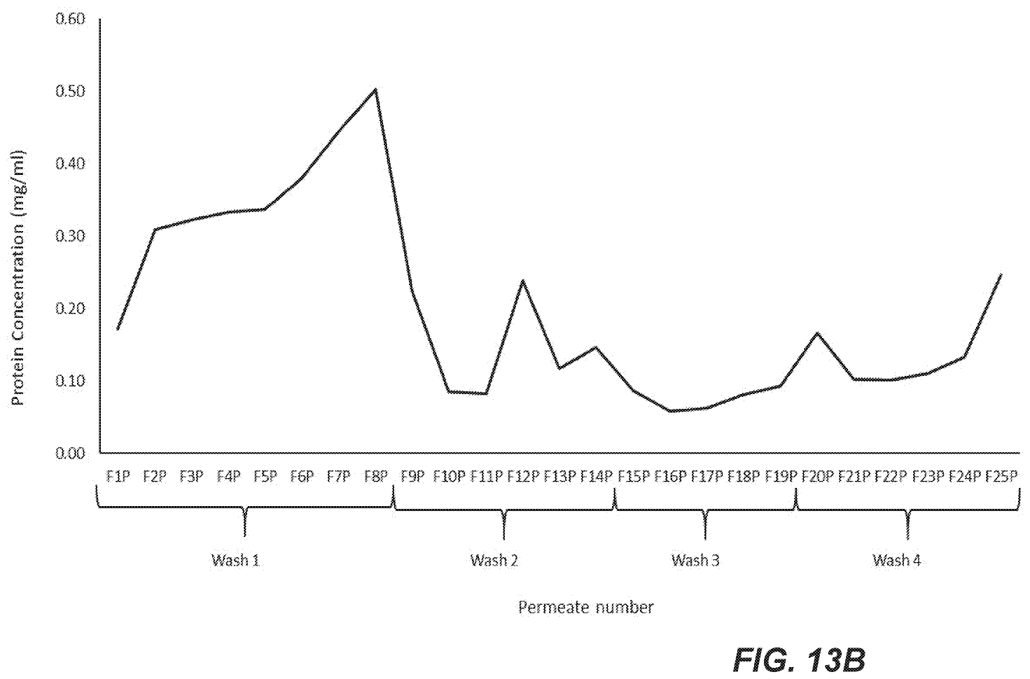

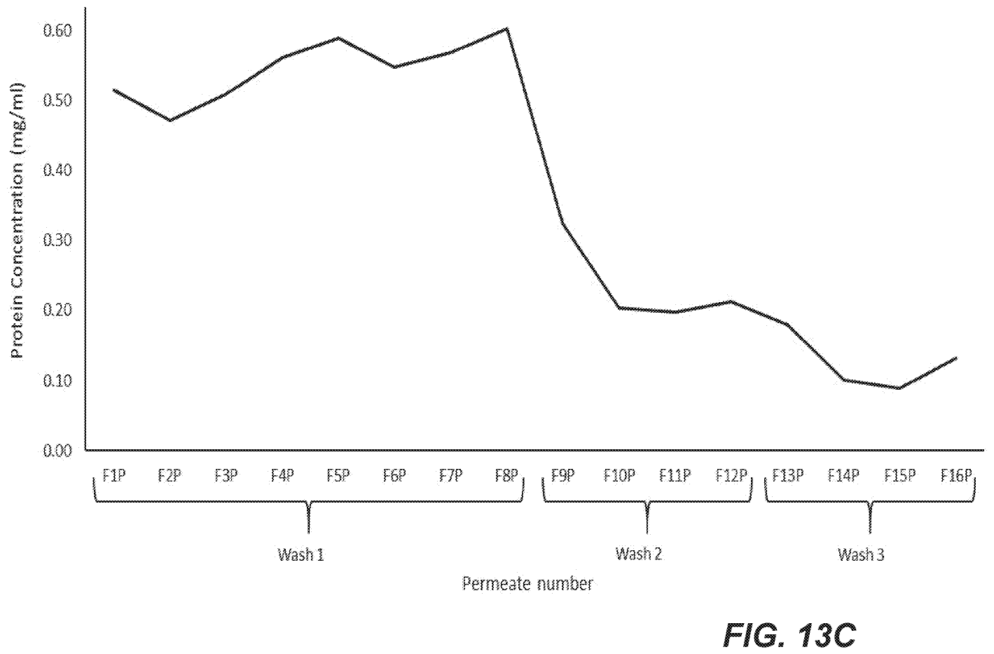

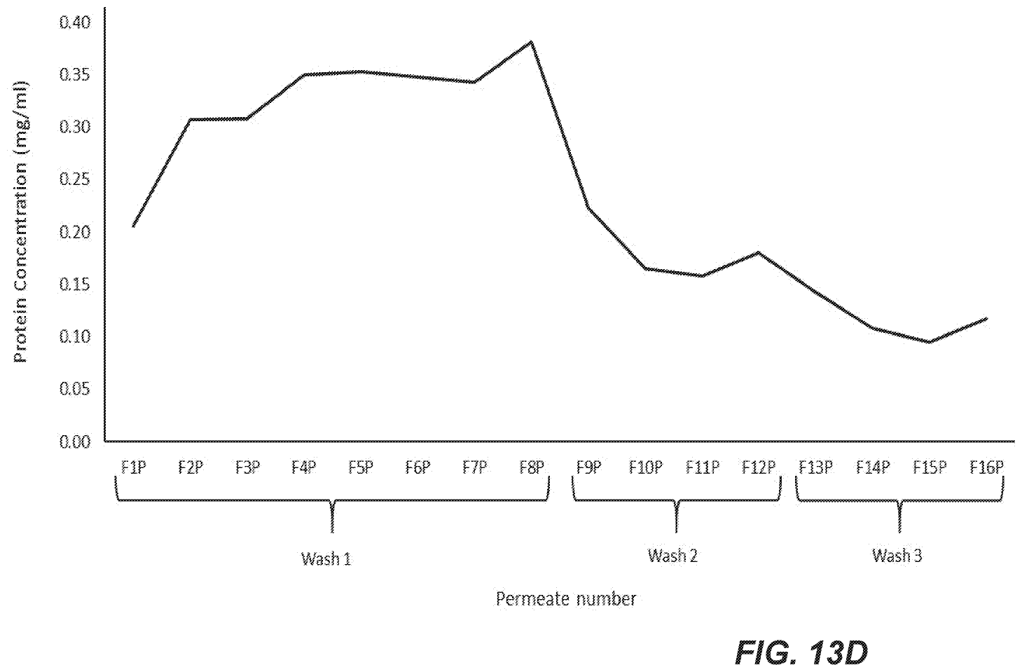

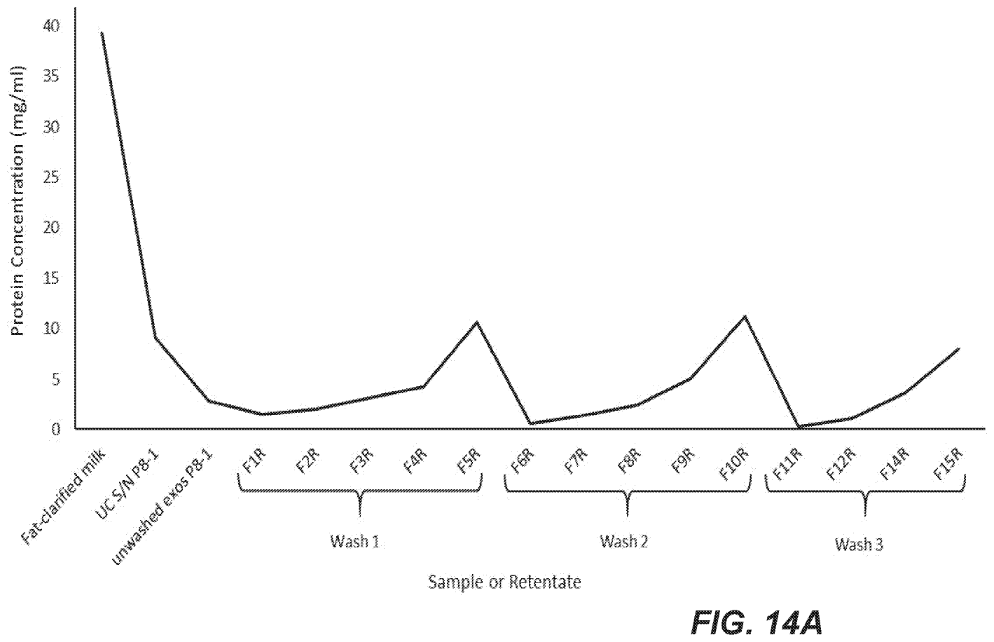

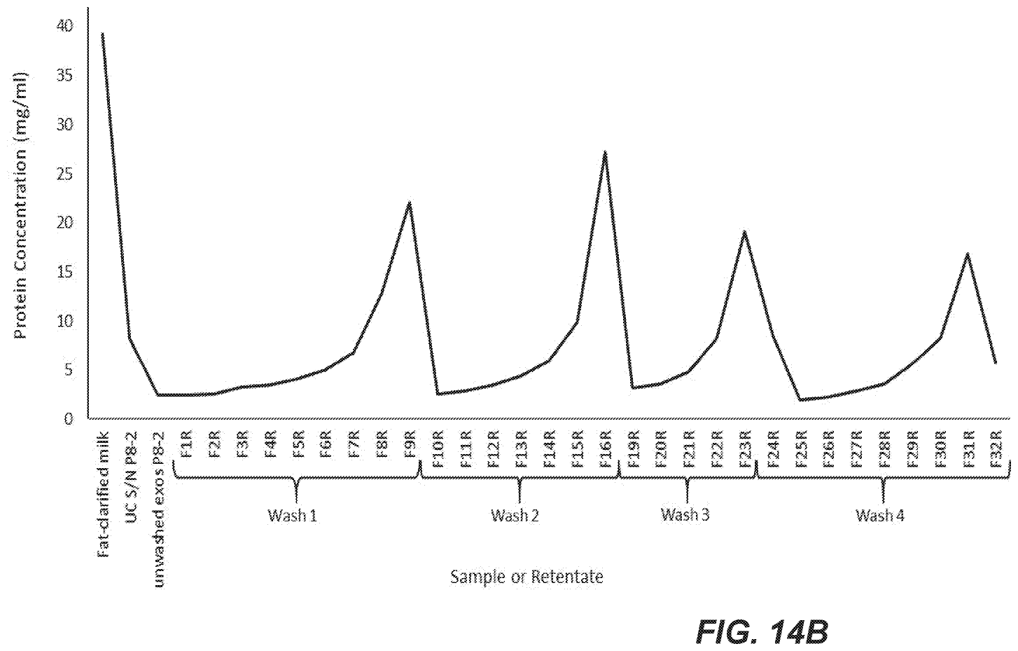

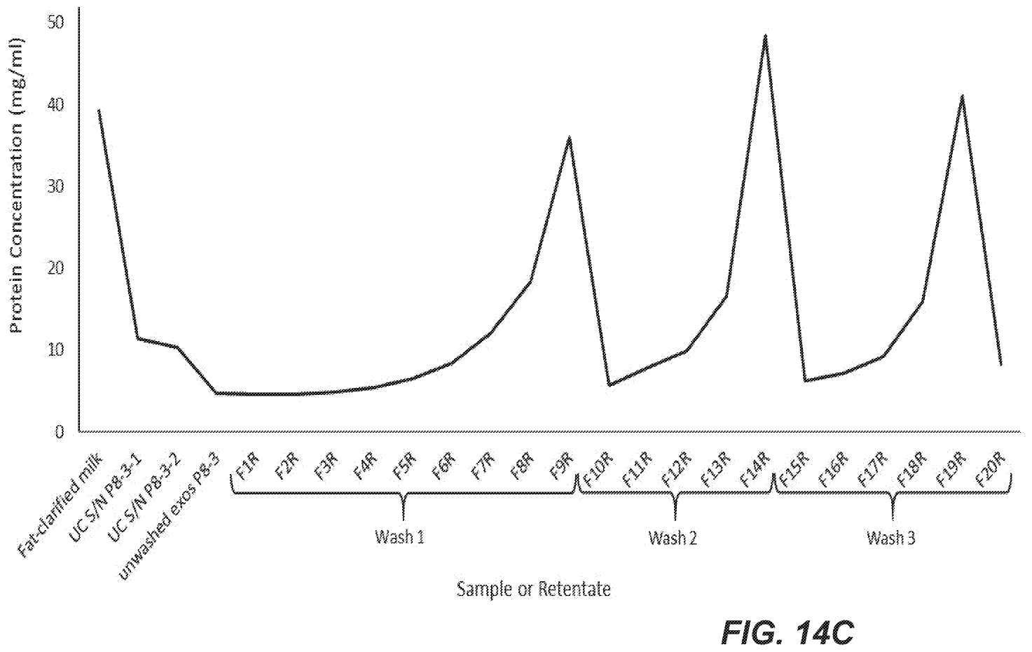

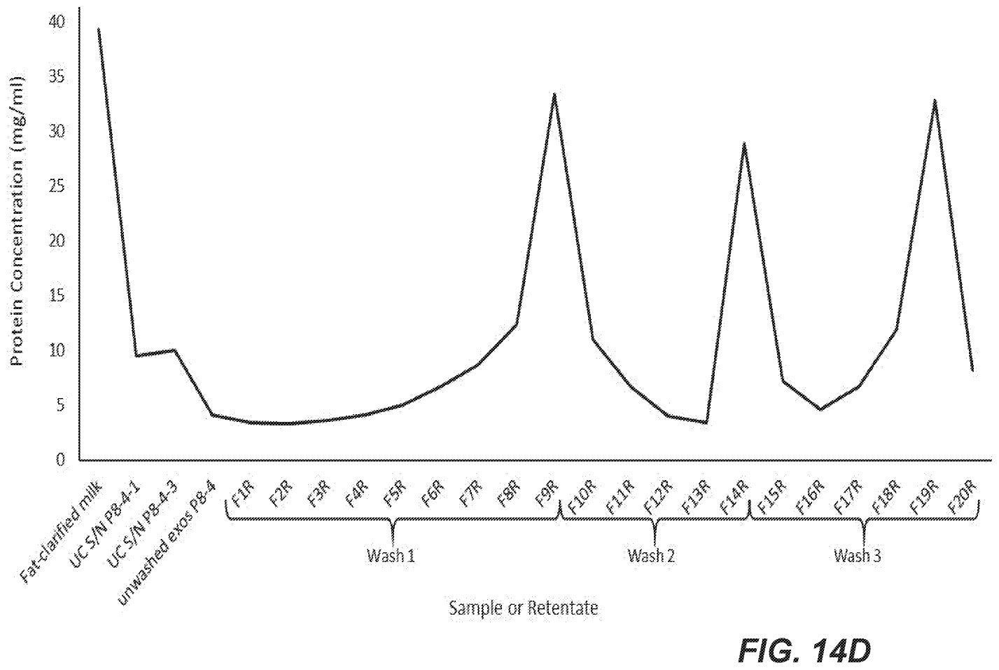

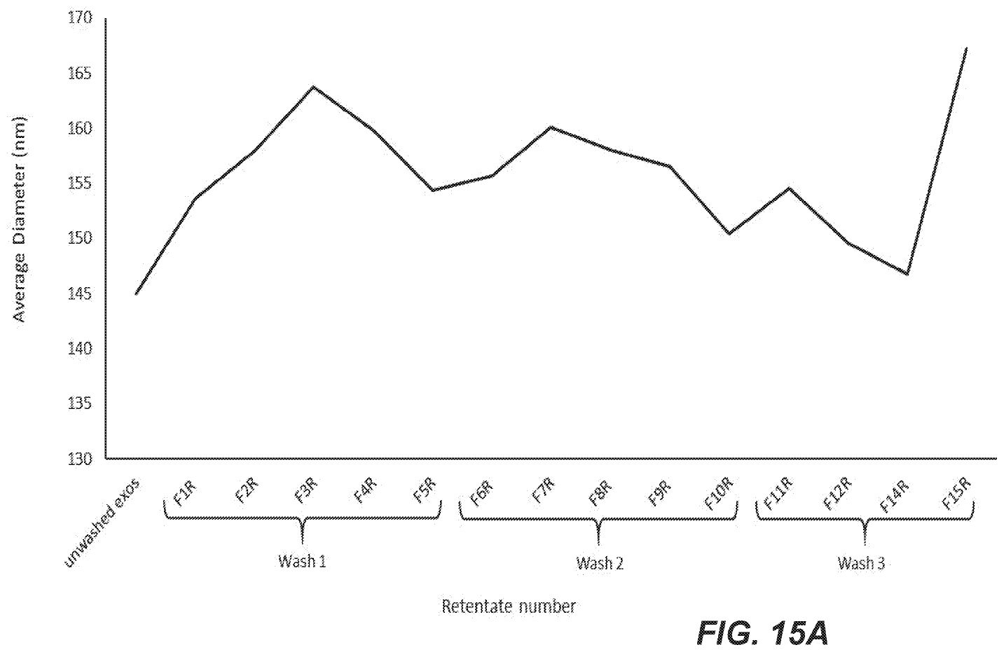

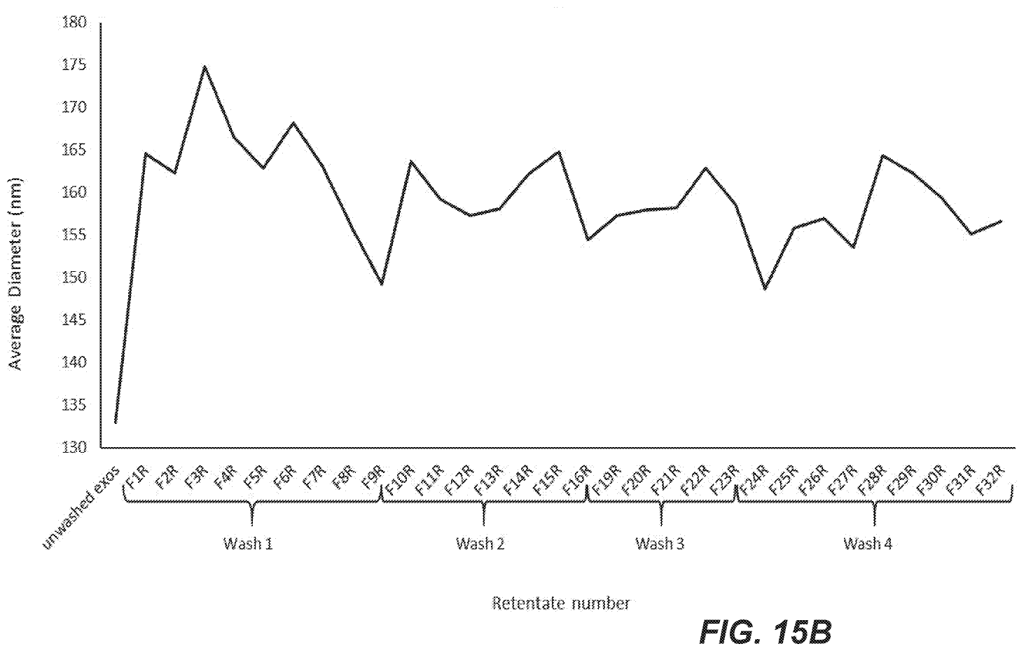

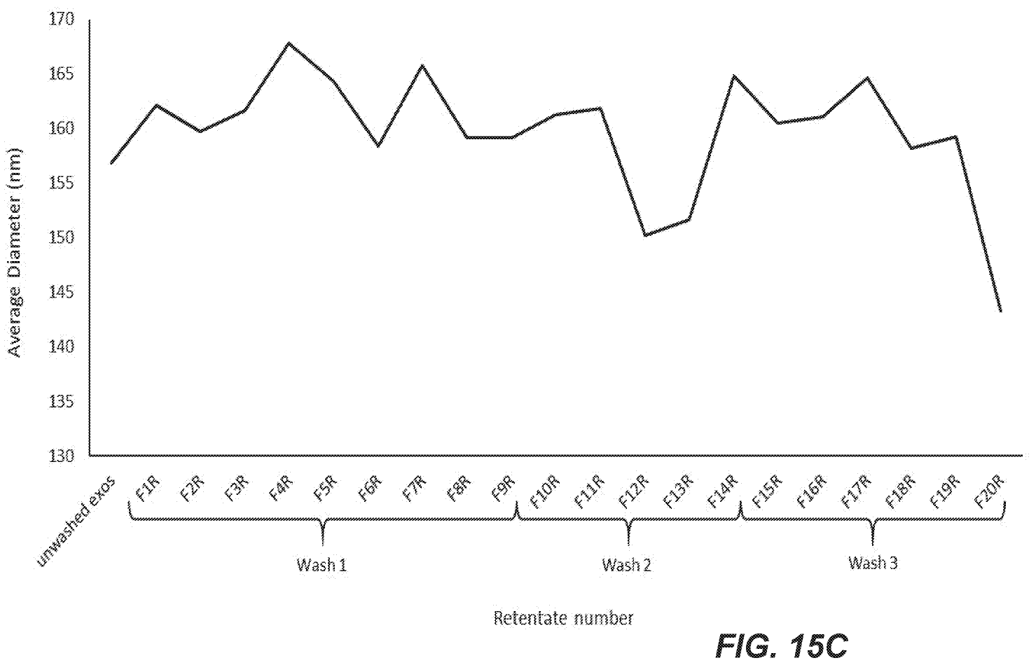

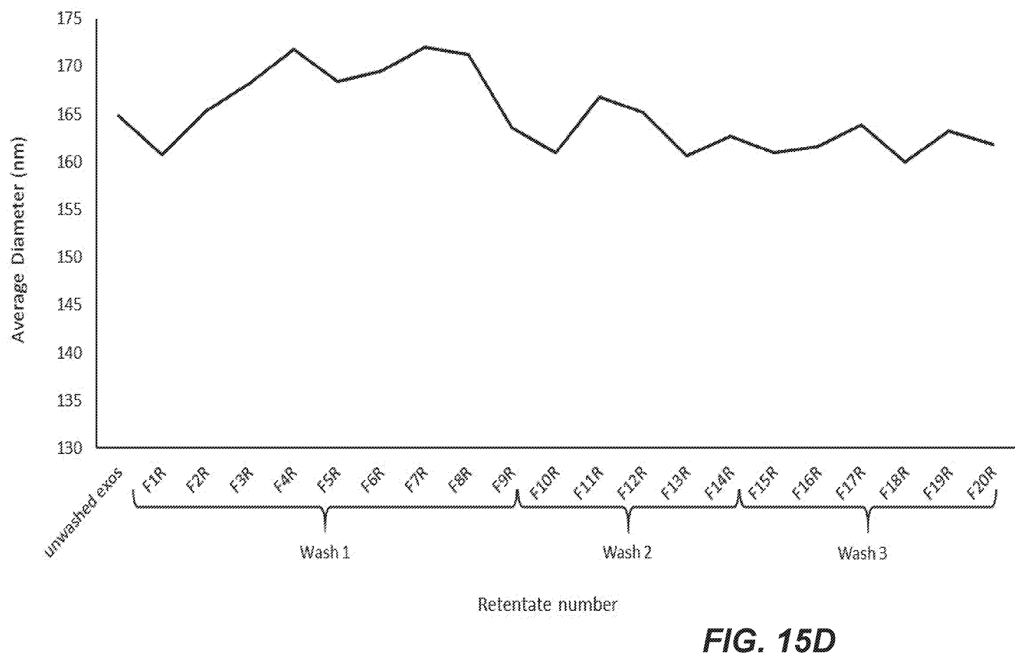

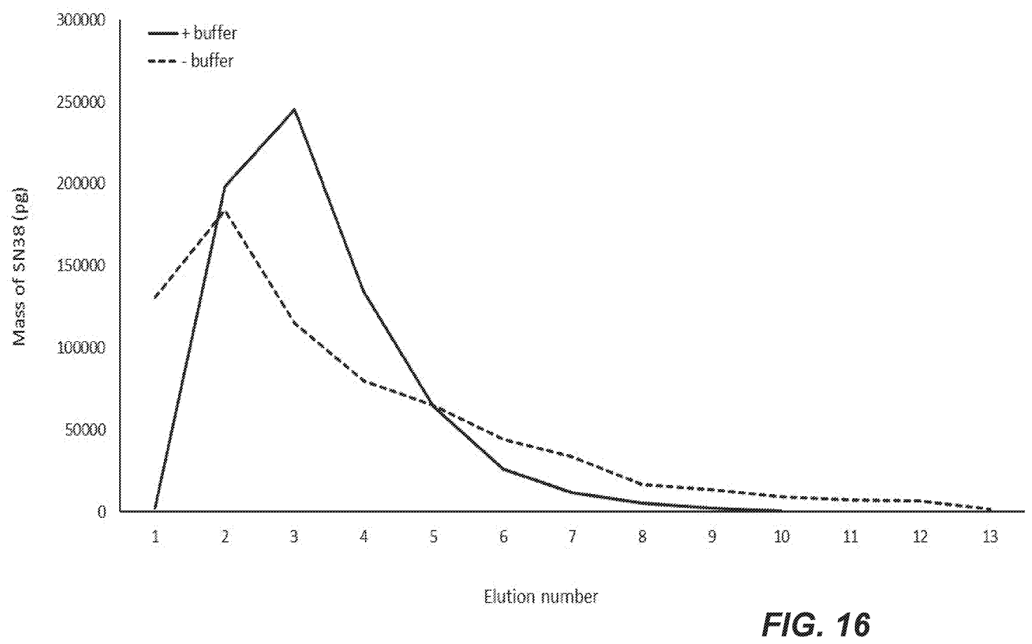

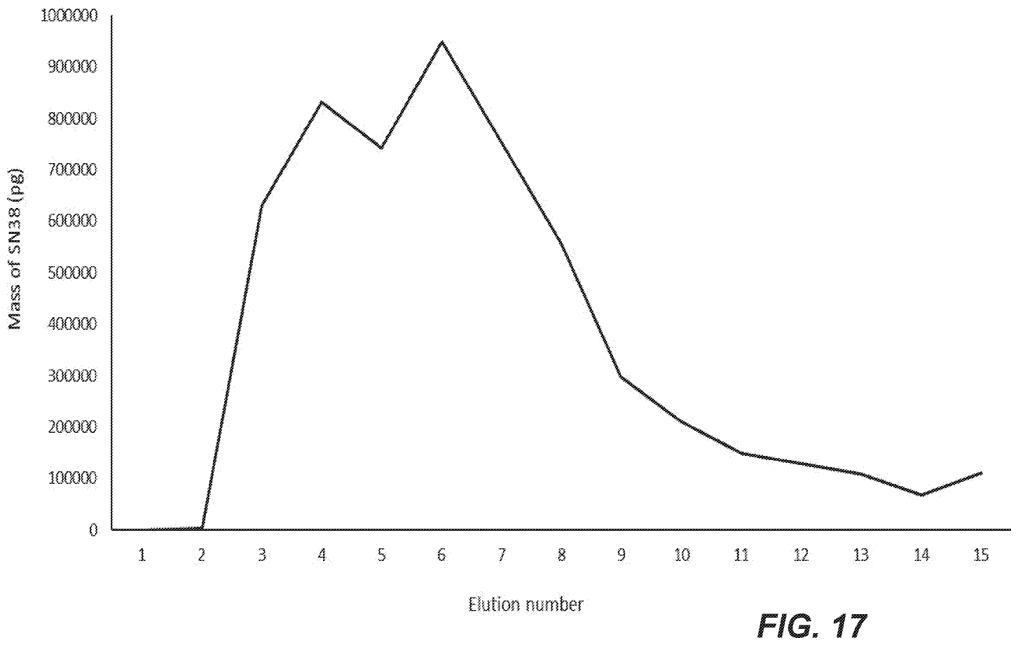

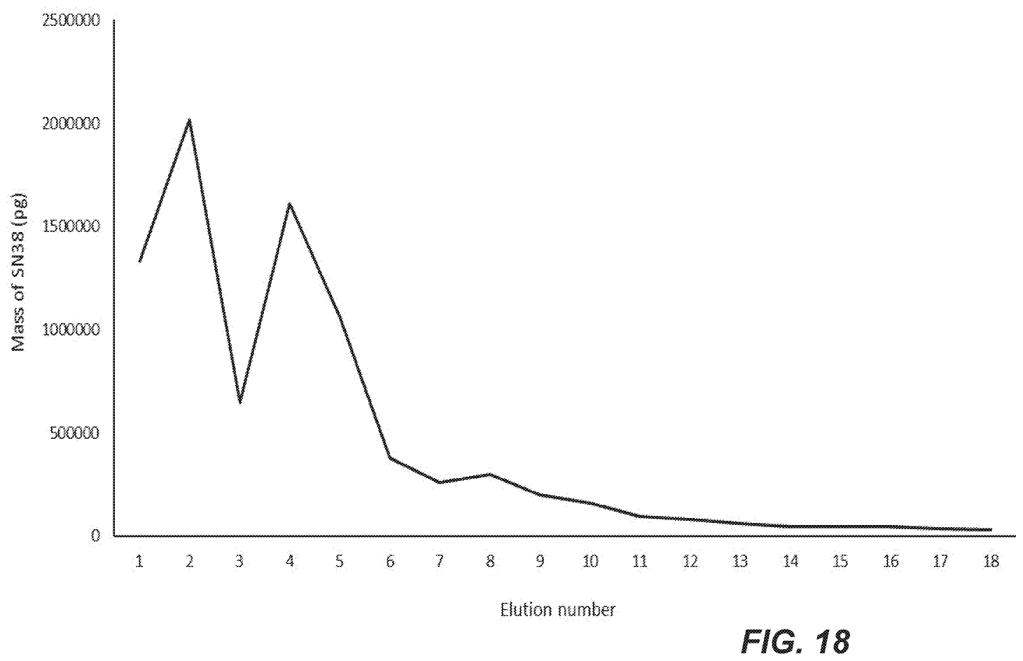

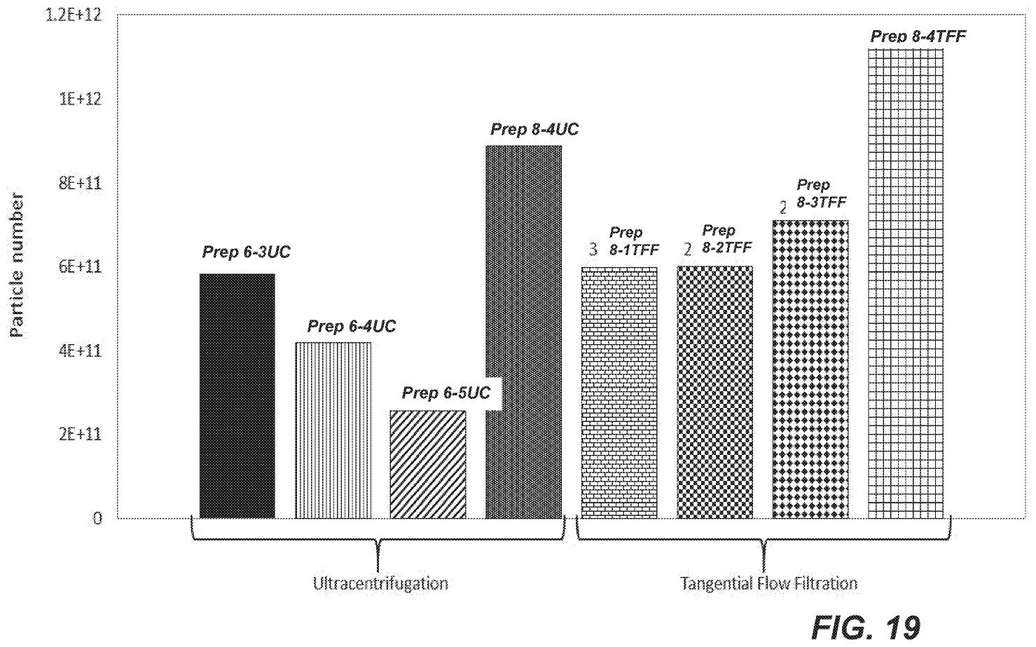

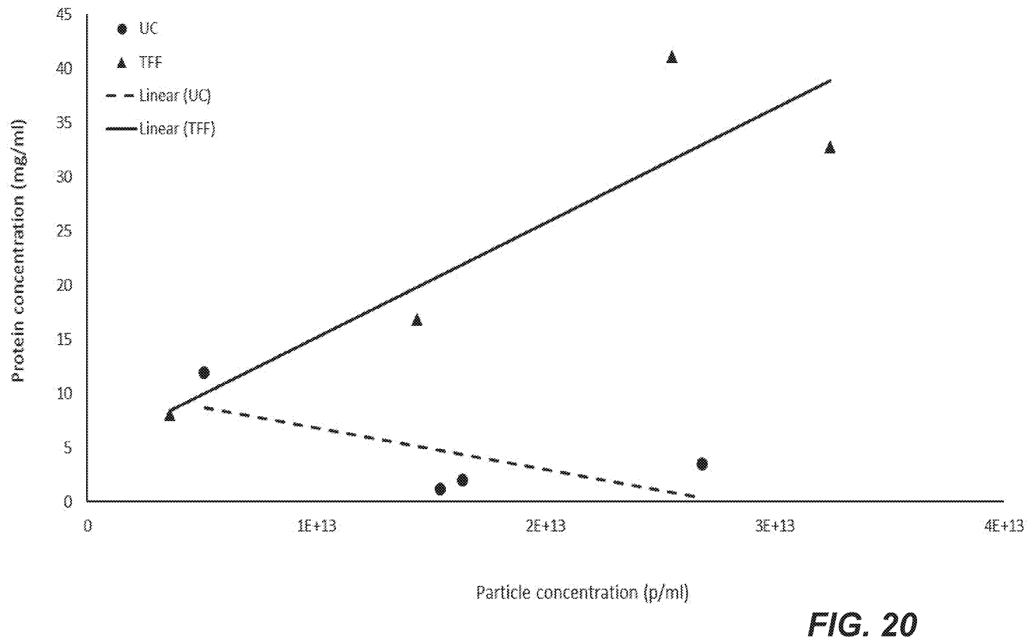

In each case, drug was incubated with exosomes for 30 min at room temperature followed by passing over a size exclusion column (Sepharose CL-4B) conditioned with 10 mM phosphate buffer, pH 5. Exosomes were eluted from the column with 150 μl 10 mM phosphate buffer, pH 5 per elution. To quantify drug by U PLC, each elution was incubated with 300 ul acetonitrile for 10 min at room temperature, and 10 mM phosphate buffer (pH 5) was subsequently added to achieve a final volume of 1.5 ml. Fifty microliters of each preparation were used for UPLC analysis. Results of the UPLC for SN38 loaded exosomes are presented in FIGS. 11-18 and Table 2. Comparisons of particles obtained using ultracentrifugation and tangential flow filtration are presented in FIGS. 19-20.

TABLE 2

| | Peak | pg | Vol | | | |
|---|---|---|---|---|---|---|
| Sample | Area | SN38 | loaded | pg/ul | ug/ul | ug in vial |
| Column 8 | 1334209 | 168631.8 | 50 | 3372.64 | 0.003373 | 5.05895 |
| E1 | | | | | | |
| Column 8 | 2018202 | 255964.9 | 50 | 5119.3 | 0.005119 | 7.67895 |
| E2 | | | | | | |
| Column 8 | 648431 | 81070.74 | 50 | 1621.42 | 0.001621 | 2.43212 |
| E3 | | | | | | |
| Column 8 | 1612850 | 204209 | 50 | 4084.18 | 0.004084 | 6.12627 |
| E4 | | | | | | |
| Column 8 | 1069132 | 134786.4 | 50 | 2695.73 | 0.002696 | 4.04359 |
| E5 | | | | | | |
| Column 8 | 378365 | 46588.36 | 50 | 931.77 | 0.000932 | 1.39765 |
| E6 | | | | | | |
| Column 8 | 260269 | 31509.7 | 50 | 630.19 | 0.00063 | 0.94529 |
| E7 | | | | | | |
| Column 8 | 299240 | 36485.57 | 50 | 729.71 | 0.00073 | 1.09457 |
| E8 | | | | | | |
| Column 8 | 202254 | 24102.27 | 50 | 482.05 | 0.000482 | 0.72307 |
| E9 | | | | | | |
| Column 8 | 161246 | 18866.32 | 50 | 377.33 | 0.000377 | 0.56599 |
| E10 | | | | | | |
| Column 8 | 97907 | 10779.11 | 50 | 215.58 | 0.000216 | 0.32337 |
| E11 | | | | | | |
| Column 8 | 82937 | 8867.72 | 50 | 177.35 | 0.000177 | 0.26603 |
| E12 | | | | | | |
| Column 8 | 62008 | 6195.48 | 50 | 123.91 | 0.000124 | 0.18586 |
| E13 | | | | | | |
| Column 8 | 47290 | 4316.27 | 50 | 86.33 | 0.000086 | 0.12949 |
| E14 | | | | | | |
| Column 8 | 44473 | 3956.59 | 50 | 79.13 | 0.000079 | 0.11870 |
| E15 | | | | | | |
| Column 8 | 48262 | 4440.37 | 50 | 88.81 | 0.000088 | 0.13321 |
| E16 | | | | | | |
| Column 8 | 37208 | 3028.98 | 50 | 60.58 | 6.06E−05 | 0.09087 |
| E17 | | | | | | |
| Column 8 | 31496 | 2299.67 | 50 | 45.99 | 4.6E−05 | 0.06899 |
| E18 | | | | | | |

The quantity of SN38 drug recovered in vial was compared against the 600 μg of protein to determine a loading percentage of 5.23% and a percent drug recovered of 47%.

### Example 8

### Loading Nanoparticles with Doxorubicin

Exosomes were isolated by differential ultracentrifugation as in Munagala et al 2016. Bovine milk-derived exosomes for drug delivery. Cancer Letter, 371:48-61. The exosomal protein concentration was determined by BCA assay, and the total weight of exosomes was calculated by doubling the protein value (from BCA) to account for an approximate 50% content of protein in biological membranes (Smyth et al 2014).

Doxorubicin was dissolved in N-methyl pyrrolidone (NMP) and added to exosomes at a 9:1 weight ratio of exosomes to drug. To assess the effect of using a buffer in the loading procedure, we compared drug loading in 10 mM phosphate buffer, pH 5 to that without the use of buffer. When buffer was added, the volume was adjusted to achieve a maximum NMP concentration of 10%.

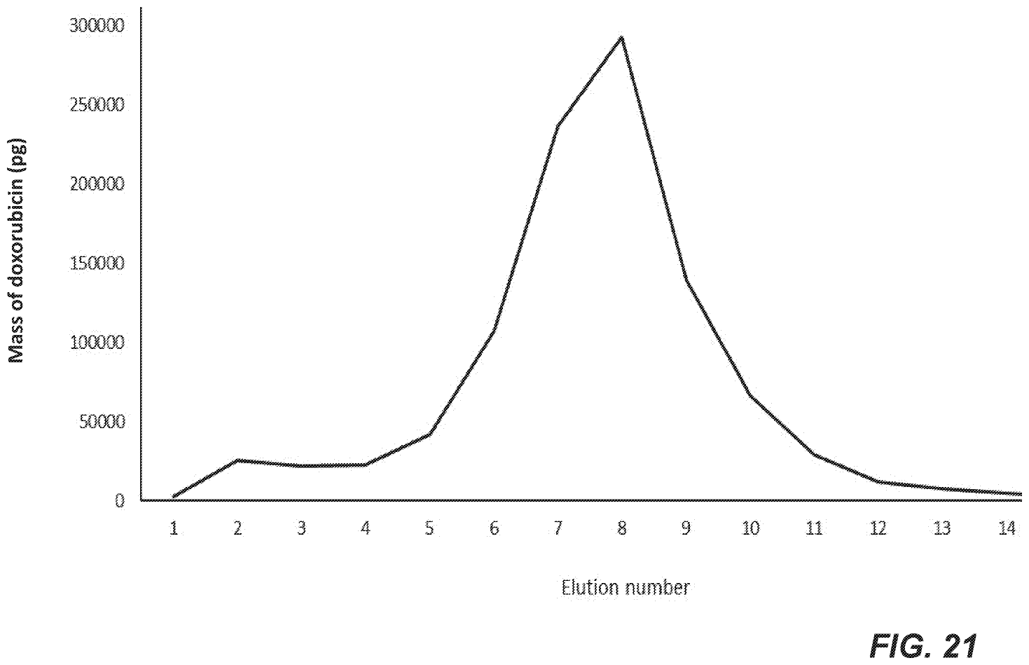

In each case, drug was incubated with exosomes for 30 min at room temperature followed by passing over a size exclusion column (Sepharose CL-4B) conditioned with 10 mM phosphate buffer, pH 5. Exosomes were eluted from the column with 150 μl 10 mM phosphate buffer, pH 5 per elution. To quantify drug by UPLC, each elution was incubated with 300 ul acetonitrile for 10 min at room temperature, and 10 mM phosphate buffer (pH 5) was subsequently added to achieve a final volume of 1.5 ml. Fifty microliters of each preparation were used for UPLC analysis. Results of the UPLC for the doxorubicin-loaded exosomes are presented in Table 3 and FIG. 21.

TABLE 3

| | Peak | Corrected | pg | Vol | | | |
|---|---|---|---|---|---|---|---|
| Sample | Area | Area | Dox | loaded | pg/ul | ug/ul | ug in vial |
| 1 | 10670 | 6060 | 2840.96 | 50 | 56.82 | 0.0000568 | 0.0852 |
| 2 | 60109 | 55499 | 26012.59 | 50 | 520.25 | 0.0005202 | 0.7803 |
| 2 | 52180 | 47570 | 22296.34 | 50 | 445.93 | 0.0004459 | 0.6689 |
| 3 | 53851 | 49241 | 23079.52 | 50 | 461.59 | 0.0004616 | 0.6924 |
| 3 | 94262 | 89652 | 42019.81 | 50 | 840.40 | 0.0008404 | 1.2606 |
| 4 | 233239 | 228629 | 107157.13 | 50 | 2143.14 | 0.0021431 | 3.2147 |
| 5 | 508689 | 504079 | 236258.18 | 50 | 4725.16 | 0.0047251 | 7.0877 |
| 6 | 629173 | 624563 | 292728.00 | 50 | 5854.56 | 0.0058545 | 8.7818 |
| 7 | 301890 | 297280 | 139333.27 | 50 | 2786.67 | 0.0027866 | 4.1799 |
| 8 | 146631 | 142021 | 66564.71 | 50 | 1331.29 | 0.0013312 | 1.9969 |
| 9 | 67373 | 62763 | 29417.17 | 50 | 588.34 | 0.0005883 | 0.8825 |
| 10 | 30740 | 26130 | 12247.59 | 50 | 244.95 | 0.0002449 | 0.3674 |
| 11 | 22063 | 17453 | 8180.76 | 50 | 163.62 | 0.0001636 | 0.2454 |
| 12 | 14822 | 10212 | 4786.96 | 50 | 95.74 | 0.0000957 | 0.1436 |
| 13 | 11264 | 6654 | 3119.36 | 50 | 62.39 | 0.0000624 | 0.0936 |
| 14 | 9996 | 5386 | 2525.058 | 50 | 50.50 | 0.0000505 | 0.0758 |

The quantity of doxorubicin drug recovered in vial was compared against the 340 μg of protein to determine a loading percentage of 8.99% and a percent drug recovered of 80.8%.

The various features and processes described above may be used independently of one another, or may be combined in various ways. All possible combinations and subcombinations are intended to fall within the scope of this disclosure. In addition, certain method or process blocks may be omitted in some implementations. The methods and processes described herein are also not limited to any particular sequence, and the blocks or states relating thereto can be performed in other sequences that are appropriate. For example, described blocks or states may be performed in an order other than that specifically disclosed, or multiple blocks or states may be combined in a single block or state. The example blocks or states may be performed in serial, in parallel, or in some other manner. Blocks or states may be added to or removed from the disclosed example embodiments. The example systems and components described herein may be configured differently than described. For example, elements may be added to, removed from, or rearranged compared to the disclosed example embodiments.

Conditional language used herein, such as, among others, “can,” “could,” “might,” “may,” “e.g.,” and the like, unless specifically stated otherwise, or otherwise understood within the context as used, is generally intended to convey that certain embodiments include, while other embodiments do not include, certain features, elements, and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any embodiment. The terms “comprising,” “including,” “having,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

While certain example embodiments have been described, these embodiments have been presented by way of example only and are not intended to limit the scope of the inventions disclosed herein. Thus, nothing in the foregoing description is intended to imply that any feature, characteristic, step, module, or block is necessary or indispensable. Indeed, the novel methods and systems described herein may be embodied in a variety of other forms; furthermore, various omissions, substitutions and changes in the form of the methods and systems described herein may be made without departing from the spirit of the inventions disclosed herein. The accompanying claims and their equivalents are intended to cover such forms or modifications as would fall within the scope and spirit of certain of the inventions disclosed herein.

What is claimed is:

1. A nanoparticle encapsulating a therapeutic agent that crosses an epithelial or endothelial barrier by transcytosis.

2. The nanoparticle of claim 1, wherein the nanoparticle is conjugated with at least one FcRn binding partner.

3. The nanoparticle of claim 2, wherein the at least one FcRn binding partner is covalently attached to the nanoparticle.

4. The nanoparticle of claim 2, wherein the FcRn binding partner is non-covalently associated with the particle through any association selected from the group consisting of affinity interactions, metal coordination, physical adsorption, host-guest interactions, hydrophobic interactions, pi stacking interactions, hydrogen bonding interactions, van der Waals interactions, magnetic interactions, electrostatic interactions, and dipole-dipole interactions.

5. The nanoparticle of any one of claims 2-4, wherein the FcRn binding partner comprises an IgG Fc fragment.

6. The nanoparticle of any one of claims 2-5, wherein the FcRn binding partner allows the nanoparticle to bind to a complex comprising an FcRn receptor.

7. The nanoparticle of claim 6, wherein the complex comprising an FcRn receptor is present in endothelial or epithelial cells.

8. The nanoparticle of claim 6, wherein binding of the complex comprising an FcRn receptor allows the nanoparticle to cross a cell layer by transcytosis.

9. The nanoparticle of claim 6, wherein binding of the complex comprising an FcRn receptor allows the nanoparticle to cross an epithelial or endothelial barrier by transcytosis.

10. The nanoparticle of any one of claims 1-9, wherein the therapeutic agent is a diagnostic, prognostic, or prophylactic agent.

11. The nanoparticle of any one of claims 1-9, wherein the therapeutic agent is selected from the group consisting of peptides, proteins hormones, cytokines, interferons, antibodies, antibody fragments, and enzymes.

12. The nanoparticle of any one of claims 1-9, wherein the therapeutic agent is selected from the group consisting of anti-cancer and anti-bacterial agents.

13. The nanoparticle of any one of claims 1-9, wherein the nanoparticle is suitable for oral administration.

14. The nanoparticle of any one of claims 1-13, wherein the particle is an exosome.

15. The nanoparticle of claim 14, wherein the exosome is isolated from bovine milk or colostrum.

16. The nanoparticle of any one of claims 1-13, wherein the nanoparticle is a liposome, a lipoplex, a micelle, or a reverse micelle.

17. The nanoparticle of any one of claims 1-13, wherein the nanoparticle is a polymeric nanoparticle.

18. The particle of claim 17, wherein the polymeric nanoparticle comprises a polymer selected from the group consisting of polyalkylenes, polycarbonates, polyanhydrides, polyhydroxyacids, polyfumarates, polycaprolactones, polyamides, polyacetals, polyethers, polyesters, poly(orthoesters), polyvinyl alcohols, polyurethanes, polyphosphazenes, polyacrylates, polymethacrylates, polycyanoacrylates, polyureas, polystyrenes, polyamines, poly(arylates), polycarbonates, polypropylene fumarates), polyhydroxyalkanoates, polyketals, polyesteramides, poly(dioxanones), polyhydroxybutyrates, polyhydroxyvalyrates, polyorthocarbonates, polyvinyl pyrrolidone), polyalkylene oxalates, polyalkylene succinates, poly(malic acid), poly(methyl vinyl ether), and poly(maleic anhydride).

19. The nanoparticle of any one of claims 1-18, wherein the nanoparticle is less than 1000 nm in diameter.

20. The nanoparticle of any one of claims 1-19, wherein the nanoparticle comprises a targeting ligand that directs the nanoparticles to a systemic target.

21. The nanoparticle of claim 20, wherein the targeting ligand is selected from the group consisting of selectins, integrins, immunoglobulins, cadherins, Arg-Gly-Asp (RGD), Asn-Gly-Arg (NGR), cyclic NGR, disulfide-based cyclic RGD (iRGD), Lyp-1, gastrin, bombesin, octreotide, epidermal growth factor (EGF), anti-EGFR antibody, vascular endothelial growth factor (VEGF), anti-VEGFR antibody, anti-HER2 antibody, hepatocyte growth factor receptor (HGFR), anti-HGFR antibody, tumor necrosis factor (TNF), or anti-TNF antibody.

22. A composition comprising a nanoparticle of any one of claims 1-21.

23. The composition of claim 22, formulated for oral administration to a mammalian subject.

24. The composition of claim 22 or 23, comprising an external phase to the nanoparticles, wherein the external phase contains less than 10%, or less than 5%, or less than 2%, or less than 1%, or less than 0.1%, or less than 0.01% unconjugated FcRn binding partners.

25. The composition of any one of claims 22-24, comprising a liquid phase external to the nanoparticles, wherein the external phase contains less than 10%, or less than 5%, or less than 2%, or less than 1%, or less than 0.1%, or less than 0.01% unencapsulated or unbound therapeutic agent(s).

26. The composition of any one of claims 22-25, comprising a liquid phase external to the nanoparticles, wherein the external phase contains less than 10%, or less than 5%, or less than 2%, or less than 1%, or less than 0.1%, or less than 0.01% protein.

27. The composition of any one of claims 22-26, comprising a liquid phase external to the nanoparticles, wherein the external phase contains less than 10%, or less than 5%, or less than 2%, or less than 1%, or less than 0.1%, or less than 0.01% Bovine Serum Albumin (BSA).

28. The composition of any one of claims 22-27, comprising a liquid phase external to the nanoparticles, wherein the external phase contains less than 10%, or less than 5%, or less than 2%, or less than 1%, or less than 0.1%, or less than 0.01% fats.

29. The composition of any one of claims 22-28, comprising a solvent selected from n-methylpyrrolidone (NMP), an alcohol, or combinations thereof.

30. The composition of any one of claims 22-29, wherein the pH of the external phase is between pH 4.5 and pH 6.5.

31. A method of producing a therapeutic effect in a subject comprising orally administering a nanoparticle of any one of claims 1-30 to the subject.

32. A method of preparing a nanoparticle of claim 1, comprising:
providing a nanoparticle comprising a therapeutic agent to be delivered encapsulated in a lipid or polymeric matrix; and,
conjugating at least one FcRn binding partner to a surface of the nanoparticle.

33. A method of preparing a nanoparticle of claim 1, comprising:
providing a nanoparticle conjugated to at least one FcRn binding partner on a surface of the nanoparticle; and,
loading a therapeutic agent into the nanoparticle.

34. A method of preparing a nanoparticle formulation, comprising:
isolating an exosome from bovine milk or colostrum;
purifying the exosome;
forming a composition comprising an external phase.

35. The method of claim 34, wherein the isolating comprises at least one of centrifugation, filtration, size selection purification, and differential binding purification.

36. The method of claim 35, wherein the size selection purification comprises size selection column purification.

37. The method of claim 35, wherein the differential binding purification comprises selection by FcRn receptor binding.

38. The method of claim 35, wherein the purifying comprises reducing any unconjugated FcRn binding partners to less than 10%, or less than 5%, or less than 2%, or less than 1%, or less than 0.1%, or less than 0.01% in the external phase of the composition.

39. The method of claim 35, wherein the purifying comprises reducing any unencapsulated or unbound therapeutic agent to less than 10%, or less than 5%, or less than 2%, or less than 1%, or less than 0.1%, or less than 0.01% in the external phase of the composition.

40. The method of claim 35, wherein the purifying comprises reducing any protein to less than 10%, or less than 5%, or less than 2%, or less than 1%, or less than 0.1%, or less than 0.01% in the external phase of the composition.

41. The method of claim 35, wherein the purifying comprises reducing any Bovine Serum Albumin to less than 10%, or less than 5%, or less than 2%, or less than 1%, or less than 0.1%, or less than 0.01% in the external phase of the composition.

42. The method of claim 35, wherein the purifying comprises reducing any fat to less than 10%, or less than 5%, or less than 2%, or less than 1%, or less than 0.1%, or less than 0.01% in the external phase of the composition.

43. The method of claim 35, further comprising adjusting the pH of the external phase of the composition to a pH between pH 4.5 and pH 6.5.

44. The method of claim 35, further comprising loading at least one therapeutic agent into the purified exosome.

45. The method of claim 44, wherein the at least one therapeutic agent comprises a solvent selected from n-methylpyrrolidone (NMP), an alcohol, or combinations thereof.

46. The method of claim 46, wherein the solvent comprises n-methylpyrrolidone (NMP).

47. The method of any one of claims 44-46, wherein the at least one therapeutic agent is a chemotherapeutic agent.

48. The method of claim 47, wherein the therapeutic agent is 7-Ethyl-10-hydroxycamptothecin (SN38).
